(19) United States
(12) Patent Application Publication
(10) Pub. No.: US 2015/0025312 A1
(43) Pub. Date: Jan. 22, 2015

(54) INTRODUCTORY ASSEMBLY AND METHOD FOR INSERTING INTRACARDIAC INSTRUMENTS

(71) Applicant: Didier de Canniere, Miami, FL (US)

(72) Inventor: Didier de Canniere, Miami, FL (US)

(21) Appl. No.: 14/174,212

(22) Filed: Feb. 6, 2014

Related U.S. Application Data
(63) Continuation-in-part of application No. 13/570,347, filed on Aug. 9, 2012.
Application No. 13/570,347 is a continuation-in-part of application No. 13/442,230, filed on Apr. 9, 2012.
(60) Provisional application No. 61/574,798, filed on Aug. 9, 2011.

(51) Int. Cl. A61B 17/00; A61B 1/018; A61B 1/04; A61B 17/34; A61B 1/32

(52) U.S. Cl. CPC A61B 17/00234; A61B 17/3423; A61B 1/32; A61B 1/04; A61B 1/018; A61B 2017/3425; A61B 2017/3419; A61B 2017/3445; A61B 2017/3488; A61B 2017/3492; A61B 2017/00243; A61B 2017/0034
USPC 600/104; 600/204

(57) ABSTRACT

An introduction assembly and method for the insertion of medical instruments through a thoracic passage into a selected one of either the left or right atrium of the heart. Catheters or other instruments dedicated to performing required cardiac maneuvers are passed through an introductory sheath having a distal end disposed within the targeted atrium. Upon completion of the required cardiac maneuvers the instruments are removed from the atrium and a closure assembly is passed through the introductory sheath into a closing relation to an entry site, formed in the pericardium and corresponding atrium wall, to facilitate healing thereof. The introductory assembly and method facilitates the concurrent, operative disposition of a plurality of catheters or other instruments into the interior of the selected atrium through different thoracic passages and entry sites thereby allowing synergistic interaction between the multiple catheters in the performance of the required cardiac maneuvers.

### CLAIM OF PRIORITY

The present application is a continuation-in-part application of previously filed, now pending application having Ser. No. 13/570,347, filed on Aug. 9, 2012, which is continuation-in-part application of previously filed, pending application having Ser. No. 13/570,347, filed on Aug. 9, 2012, which is a continuation-in-part application of previously filed, pending application having Ser. No. 13/442,230, filed on Apr. 9, 2012, which is based on and a claim of priority is made under 35 U.S.C. Section 119(e) to a provisional patent application that is currently pending in the U.S. Patent and Trademark Office, namely, that having Ser. No. 61/574,798 and a filing date of Aug. 9, 2011, and which are both incorporated herein by reference.

### BACKGROUND OF THE INVENTION

1. Field of the Invention

The invention relates to intracardiac surgical procedures and more specifically to an assembly and method for introducing medical instrumentation through one or more introductory sheaths, to a predetermined intracardiac depth, into a selected one of the left atrium or right atrium through a thoracic passage and correspondingly disposed intercostal spaces. Upon completion of the required surgical procedure on the interior of the targeted atrium, a closure assembly is disposed in closing relation to the entry site of the instrumentation and introductory sheath in the pericardium and targeted atrium wall.

2. Description of the Related Art

When the heart or any of its component parts develops a defect or disease, intracardiac intervention is often necessary to correct, repair, and/or replace damaged or defected cardiac components. Classically, this has been accomplished through surgery in which the chest of the patient is opened and the heart, which is arrested and/or bypassed, is operated on. This can be a very dangerous procedure replete with many possible complications resulting from, at the very least, stopping or bypassing the heart, general anesthesia administered during the procedure, risk of infection from a large opening in the chest cavity, and scarring. Moreover, surgery is not a viable option for many elderly and/or frail patients who are at an increased risk for these complications.

A widely used alternative to cardiac surgery is invasive cardiology, in which catheters are introduced into blood vessels at remote, or peripheral, sites from the heart and are steered through veins and arteries of the body to reach the heart. For example, the femoral vessels, radial artery, subclavian artery and the jugular veins can be used for insertion of catheters for remote cardiac intervention. While this approach avoids many of the risks of surgery, it suffers from significant technical limitations. First, the anatomy and size of the peripheral vessels precludes the use of some catheters. For example, the capillaries and some veins are too narrow to accommodate catheters. Some veins may not be sufficiently sized for a larger catheter, such as in excess of 12 French, or to accept a plurality of catheters simultaneously. The suitability of blood vessels for remote cardiac access may be further exacerbated in many patients, namely the elderly, in which the vessels are narrowed, calcified or tortuous, making access to the heart difficult or impossible. Moreover, the branched network of blood vessels makes the usage of multiple catheters limited to only those catheters having a small caliber. However, even in situations such as these, maneuverability is limited since very little torque can be developed between two catheters threaded through a common blood vessel once inside the heart to address any target structure. This can involve severe limitations since many intracardiac maneuvers require complex access and steering such as, but not limited to, trans-septal punctures, steering the catheter through the inter-atrial septum to access the mitral valve, such as for delivering a MITRACLIP®, percutaneous mitral dilatation, and steering ablation catheters around the openings of the pulmonary veins.

The distance that separates the entry point of the catheter from the target structure is an additional drawback to invasive cardiac measures performed through blood vessels. Moreover, the further the distance from the remote point of entry to the heart, the further the catheter must be threaded and the greater the risk of inadvertently puncturing the wall of a blood vessel, encountering a blockage or collapsed blood vessel, or other obstacle. Moreover, long catheters are also required when the entry point is remote from the heart, necessitating an increase in materials which can become cumbersome to control and maneuver as intended.

More recently, new approaches to intracardiac structures have been introduced to deliver prostheses, such as aortic valves as in the case of transaortic valve implantation (“TAVI”), for patients who do not qualify for a classical surgical replacement and/or whose peripheral vessels are too small to accommodate the large catheters needed to carry the prosthesis. In such an approach, a direct puncture is made in the apex of the left ventricle of the heart via a small incision in the chest wall by an anterior thoracotomy. This approach is becoming more popular and is currently investigated as a route to deliver treatment for other structural heart disease such as, but not limited to auto-implantable mitral prostheses, etc.

However, this entry procedure also has recognized disadvantages. More specifically, this procedure requires general anesthesia and the indicated thoracotomy generates pain, requires long rehabilitation and in known to result in significant complications in especially frail patients. Further, it involves entering the ventricular wall, which leads to a marginal loss of contractile force of the heart, but also a significant risk of bleeding, since the pressure in the ventricle is about 10 times higher than in the atrium. It also requires passage through the ventricular trabeculae and subvalvular mitral apparatus which are needed to prevent backflow of blood during the contraction of the heart, known as systole.

It would therefore be beneficial to implement an improved and proposed introductory assembly and method of accessing the chambers of the heart and performing intracardiac interventions. Such an improved technique would not require arresting or bypassing the heart and illuminate the blood vessels used for peripheral access to the heart. As a result instrumentation including multiple catheters could be concurrently introduced into predetermined areas of the heart, specifically including the interiors of the right and left atria, in a manner which would eliminate or significantly reduce many of the complications and disadvantages of known surgical procedures.

### SUMMARY OF THE INVENTION

The present invention is directed to an introduction assembly and method for accessing intracardiac structures through the insertion of catheters or other instrumentation into either the right or left atrium. At least one puncture or entry site is formed in the targeted atrium of a beating heart, by inserting a lancet through a thoracic passage by way of an appropriate intercostal space and entering the corresponding portion of the pericardial bag surrounding the targeted atrium of the heart. It is recognized, that in some cases, accessing the atrium through the right side of the chest may be preferred. The introductory assembly and method of the present invention can be used and accomplished from any approach to the heart which enables access to the targeted atrium.

Moreover, the present invention may be used with or without lung deflation, although in some situations it may be preferable to deflate one lung, preferably the right lung, to create additional space in which to work. The present invention also has the distinct advantage of allowing a variety of intracardiac maneuvers to be performed. By way of example such intracardiac maneuvers include, but are not limited to, closing para-valvular prosthetic leaks; closing the left atrial appendage; approaching the mitral and/or tricuspid annuli and/or leaflets to deliver devices that restrain their prolapse or limit their dilatation; encircling the pulmonary veins with ablation lines performed with different energy sources, and repair or replacement of a malfunctioning atrio-ventricular valve. Further, the introductory assembly and method may be utilized surgically after a small, possibly robotically-enhanced right thoracotomy. In this case the atria are opened (“atriotomy”) to manually perform the intended intracardiac maneuver(s).

Accordingly, the present invention provides many advantages that overcome the limitations of other known ways of accessing and performing intracardiac interventions. Further by way of non-limiting examples, the practicing of the various preferred embodiments of the present invention reduces the limitations imposed by peripheral access to the heart through blood vessels, such as a narrowing of the vascular tree which precluding catheter passage. The present invention facilitates the ability to insert multiple catheters from different entry points through the thoracic wall and into a targeted atrium. This multiple, concurrent insertion capability thereby permits synergistic action, force, and/or torque between the catheters because they need not be coaxially disposed in relation to each other. This is in contrast to catheters inserted through the venous or arterial vasculature.

In addition, the present invention may be practiced under general anesthesia or sedation, advantageously with temporary single lung ventilation and/or intrapleural carbon dioxide₂ insufflation to temporarily collapse one lung if additional space is needed. The site of the puncture(s) or entry sites may be predetermined with imaging, such as 3D CT reconstruction of the cardiac structures relative to the rib cage, and may be performed in a cath lab or preferably a hybrid operating room under fluoroscopy, preferably with transoesophageal echographic guidance.

In more specific terms, the present invention includes an introduction assembly for the insertion of medical instruments such as, but not limited to, catheters through a thoracic passage and into either the right or left atrium of the heart. As such, a puncturing or cutting instrument is dimensioned and structured to form an entry site into the targeted right or left atrium by first penetrating a corresponding portion of the pericardial bag. The puncturing instrument is introduced through a thoracic passage and an appropriate intercostal space. In addition, an elongated introductory sheath or like tubular structure includes a central lumen and is movably disposed over the puncturing instrument so as to extend through the entry site formed in both the pericardial bag and the targeted right or left atrium. The sheath also includes a distal end having a predetermined “intracardiac length” which is positioned on the interior of the targeted atrium.

Additional structural features of the inserted introductory sheath include a buffer disposed thereon in segregating relation between the distal end of the sheath, which enters the targeted atrium, and the remainder of the sheath disposed exteriorly of the targeted atrium. As applied, the buffer is disposed in confronting disposition with an exterior portion of the pericardial bag, which corresponds to the entry site. As such, the buffer may be at least partially determinative of the intracardiac length. More specifically, the spacing of the buffer from the extremity of the distal end disposed into the targeted atrium through the entry site may determine the intracardiac length. Therefore, the intracardiac length may be considered the length of the distal end of the sheath which is allowed to pass into the targeted atrium. The central lumen of the introductory sheath is dimensioned and configured to receive and facilitate passage therethrough of instrumentation, such as catheters, which are dedicated to the performance of the intended or predetermined cardiac maneuvers within the targeted atrium. Subsequent to the completion of the intended cardiac maneuvers within the selected atrium, a closure assembly is disposable in an operative position in closing relation to the entry site formed in both the pericardial bag and the atrium wall of the targeted atrium.

Yet another embodiment of the present invention includes additional structure which facilitates the establishment and maintenance of the intended and appropriate intracardiac length of the distal end of the sheath within the targeted atrium, while also preventing the inadvertent removal of the distal end from the targeted atrium. More specifically, one or more preferred embodiments of the present invention include a restricting assembly connected to the distal end of the sheet and movable therewith into and out of the targeted atrium through the entry site. Moreover, the restricting assembly is selectively disposable into a collapsed or reduced size orientation or a restricting orientation. Accordingly, the restricting assembly may be in the form of a collapsible and/or inflatable bladder connected to the distal end and extendable outwardly therefrom when positioned inside the targeted atrium. Further, the dimension and configuration of the bladder, when in the restricting orientation, is sufficient to prevent and/or significantly restrict the removal of the distal end from the interior of the targeted atrium. In contrast, when the expandable or inflatable bladder is in the collapsed orientation it assumes a size and configuration which facilitates or at least allows its passage, along with the distal end, through the entry site into and out of the interior of the targeted atrium. As set forth in greater detail hereinafter, the restricting assembly specifically, but not exclusively, comprising the expandable or inflatable bladder may be used in combination with or independently of the various embodiments of the buffer, as also more specifically described hereinafter. Further, in that the bladder of the restricting assembly is structured to be selectively inflated and deflated, a pressurized fluid source may be connected in fluid communication with the interior of the bladder and facilitate it being inflated and/or deflated as desired. The fluid source may be located exteriorly of the sheath and the thoracic passage and operated independently. Moreover, any of a variety of different fluid communicating connections may be established between the pressurized fluid source and the inflatable bladder.

In one or more preferred embodiments, the buffer comprises or is directly associated with a securing assembly which includes a vacuum or negative pressure source. The buffer is connected to the vacuum source preferably through one or more lumens, flow lines, conduits or other appropriate structures connected to or mounted on the introductory sheath. As such, fluid communication is established between the buffer and the vacuum source to the extent that and appropriate negative pressure is developed and communicated to the buffer through the flow lines. The negative pressure is sufficient to removably secure the buffer to the exterior surface of the pericardial bag in appropriately adjacent relation to the entry site formed in both the pericardial bag and the atrium wall.

Yet additional structural and operative features of at least one preferred embodiment of the buffer include it having an at least partially collapsible construction. Moreover, the collapsible construction of the buffer may comprise a plurality of pads extending outwardly from the exterior of the sheath into a disposition which facilitates the aforementioned removable securement to the exterior of the pericardial bag adjacent to the entry site. In yet another preferred embodiment, the buffer may include an annular configuration connected to and at least partially surrounding exterior portions of the sheath. As such, the buffer is extendable transversely outward from the sheath into the aforementioned removable securement. Therefore, by the application of the negative pressure or vacuum associated with the buffer, the introductory sheath is disposed in movement restricting relative to the entry site. As should be noted, the regulation of fluid flow between the vacuum source and the buffer will allow control over the attachment of detachment of the buffer from its stabilized position relative to the pericardial bag.

Yet additional structural and operative features of at least some of the preferred embodiments of the present invention include the aforementioned closure assembly. More specifically, the closure assembly may comprise a first segment and a second segment respectively and concurrently disposable interiorly and exteriorly of the entry site. As such, the first segment of the closure assembly passes through the lumen of the introductory sheath, through the entry site and into the interior of the targeted atrium. Cooperatively, the second segment of the closure assembly also passes through the lumen of the introductory sheath and is disposed exteriorly of the pericardial wall and entry site. Interconnecting structure between the first and second segments of the closure assembly may be operatively manipulated such as from an exterior of the proximal end of the introductory sheath. Such manipulation of the interconnecting structure will bring the first and second segments to closing relation to the entry site as they are respectively disposed on the interior of the targeted atrium and on the exterior of the pericardial wall. When disposed in the intended sealing relation to the entry site, the first and second segments will effectively “sandwich” the entry site therebetween and facilitate its closure.

The segments of the closure assembly may be formed of a material which will dissolve within the time required for the healing of the entry site. Moreover, the first and second segments of the closure assembly are also formed of a collapsible material which has an at least minimal inherent bias. These collapsible characteristics allow the folding or sufficient size reduction of the first and second segments to pass through the introductory sheath to the entry site. However, upon passage from the open distal end of the lumen of the introductory sheath, each of the first and second segments will be automatically expanded into an intended operative size and configuration for their respective disposition into closing relation to the entry site.

The relative fragile nature of the atrium wall and the possibility of relative displacement or movement between the pericardial bag and the atrium is recognized in the medical arts. Accordingly, additional preferred embodiments of the present invention include at least a portion of sheath being formed of a flexible material. Moreover, the flexible material portion of the sheath should be structured to demonstrate sufficient and/or a predetermined minimum amount of flexibility to accommodate relative movement between the pericardial bag and at least the corresponding wall of the targeted atrium adjacent to the entry site. In addition, this predetermined amount of flexibility should be sufficient to eliminate or significantly reduce the possibility of tearing, ripping or like damage being done to the relatively fragile wall tissue of the targeted atrium. Absent this sufficient flexibility in the distal end, as well as a length of the sheath 50 extending from the distal end and engaging, passing through and/or correspondingly disposed with the entry site, damage of the targeted atrium wall may occur. Such damage may be the result of, but not limited to, forces placed on the atrium wall 44 and/or pericardium bag by a non-flexible portion of the sheath which passes through or is sufficiently close to the entry site and/or which is disposed within the targeted atrium, when relative movement or displacement occurs between the pericardium bag and the atrium wall.

As set forth above, the various preferred embodiments of the present invention are directed not only to the introduction assembly, as generally set forth above, but also to a method of introducing medical instrumentation through a thoracic passage and into a targeted one of the either the right or left atrium of the heart. Accordingly, in cooperation with the introductory assembly as set forth above, the method of at least one preferred embodiment of the present invention comprises the forming of at least one entry site into the targeted atrium and into a corresponding part of the pericardial wall. The aforementioned introductory sheath is positioned such that a distal end thereof, having the predetermined intracardiac length, extends through the thoracic passage and the entry site into the targeted atrium along a predetermined length. Once so positioned, appropriate instrumentation, such as catheters, dedicated to perform the intended predetermined cardiac maneuvers, are passed along the interior of the introductory sheath and into the targeted atrium through the entry site. Once the predetermined cardiac maneuvers have been completed the instrumentation is removed from the selected atrium through the introductory sheath. Thereafter the aforementioned closure assembly is passed through the central lumen of the introductory sheath and into a closing relation with the entry site.

The introduction assembly of the present invention includes yet another preferred embodiment comprising a plurality of operative components cooperatively structured to facilitate the introduction of instrumentation into the interior of the heart. In addition, this additional embodiment is operative to introduce the instrumentation along a thoracic passage and through the chest cavity. This is distinguishable from introductory systems, specifically including introduction catheters or like structures having a sufficiently reduced size to pass through blood vessels into the heart. Accordingly, the introduction assembly of this additional embodiment facilitates the introduction of comparatively large instrumentation including, but not limited to, replacement valves having improved structural integrity and/or features which facilitate an effective replacement of the organic valve, when necessary.

In more specific terms, this embodiment of the introduction assembly includes an elongated sheath preferably formed of a rigid material such as, but not limited to, stainless steel. The sheath is longitudinally dimensioned to be accurately disposed along the thoracic passage for operative placement relative to an exterior of the heart wall. Moreover, the sheath includes an open proximal end typically disposed exteriorly of the thoracic passage so as to provide access thereto and the introduction of medical instrumentation there through. In addition the sheath includes an open distal end which preferably disposed in substantially flush, confronting relation to the exterior of the heart wall when operatively placed. In addition, a portion of the length of the sheath preferably extending from the open distal end outwardly towards the open proximal end, but in spaced relation thereto, comprises a predetermined configuration. This predetermined configuration, along with the rigidity of the sheath, facilitates the aforementioned orientation and disposition of the open distal end into the substantially flush confronting relation of the open distal end with the exterior of the heart wall adjacent an entry site through the heart wall.

The aforementioned predetermined configuration of at least a portion of the length of the sheath is more specifically defined by a curved configuration extending along a portion of the length thereof generally from the open distal, as set forth above. The degree of curvature and the length of the predetermined curved portion of the sheath may be varied when forming different sheath structures. More specifically, the amount of curvature and the length of the curved configuration may be dependent, at least in part, on the introductory site of the sheath and the thoracic passage along which the sheath is manipulated as it approaches and reaches the heart. Therefore, the rigidity of the sheath is such as to significantly enhance the ability to accurately and reliably manipulate the sheath along the intended thoracic passage. As a result, the positioning of the open distal and into the aforementioned flush, confronting relation to the exterior of the heart wall will accurately and reliably accomplished. This in turn will enhance the ability to position and form the entry site into the heart at a precise, intended location.

It is recognized that the type of instrumentation introduced utilizing this additional preferred embodiment of the present invention will be dependent upon the medical procedure required to be performed. By way of example only, such instrumentation may include replacement components associated with the mitral valve, tricuspid valve, etc.; instrumentation operative for the occlusion of the left atrial appendage; instrumentation intended to correct and/or repair the leaflets of the mitral valve in order to prevent regurgitation of blood back into the left atrium. Therefore, the diameter or internal transverse dimension of the sheath, as well as other operative components of the introduction assembly is preferably greater than 7.3 mm and could be significantly greater, such as in the range between 7.3 mm and 1.5 cm.

Additional components of the introduction assembly of this embodiment of the present invention includes a stabilizer assembly introduced into the heart through the interior of the sheath. Further, the stabilizer assembly includes an elongated catheter having a central lumen and a stabilizer structure secured to the distal end thereof. The stabilizer structure is disposed through the entry site, once the entry site is dilated. Also, the stabilizer structure includes a central channel or passage disposed in direct fluid communication with the central lumen of the stabilizer catheter. When operatively disposed, the stabilizer structure is removably anchored or retained within the entry site so as to maintain it in the expanded orientation. As a result, the introduction of the instrumentation into the interior the heart, through the central channel or passage of the stabilizer structure, is accurately and reliably accomplished.

Accordingly, both the stabilizer catheter and the stabilizer structure secured thereto include a generally enlarged diameter or internal transverse dimension of the central lumen and central passage respectively. While the stabilizer catheter and the stabilizer structure pass telescopically within the interior of the aforementioned sheath, both include the aforementioned expanded interior diametrical dimension, which also may be in the range of 7.3 mm to 1.5 cm.

Also, in order to removably retain the stabilizer structure within the expanded entry site, the stabilizer structure includes a retaining assembly. The retaining assembly preferably includes at least two retaining members each disposed in a different opposite end of the retaining structure in surrounding relation to the central passage thereof. Each of the two retaining members may be independently activated so as to assume a retaining orientation. In at least one preferred embodiment, each of the retaining members may be independently and selectively inflated and deflated in order to retain the stabilizing structure within the expanded entry site and allow it to be removed there from upon completion of the intended medical procedure. Accordingly, when operatively positioned and activated the two retaining members are respectively disposed on the interior and exterior of the heart, preferably in confronting, retaining relation to interior and exterior surfaces of the heart wall.

As indicated, the effective and accurate disposition of the stabilizer assembly is accomplished by passage it telescopically within the interior of the sheath and telescopically exterior of a dilator. In use, the dilator is first introduced through the interior the sheath, where in the stabilizer assembly is telescopically movable on the exterior of the dilator and on the interior of the sheath. The dilator is then positioned into alignment with the entry site, formed by a penetrating needle, and force there through in a manner which serves to dilate the entry site into a desired size.

More specifically, in forming the entry site the penetrating needle is first introduced through the interior of the sheath and is forced through the wall of the heart thereby first defining the entry site. Once so positioned on the interior of the heart a guide wire passes through the interior of the penetrating needle and out through an open, interior end thereof. Once the guide wire is introduced into the interior of the heart, the penetrating needle is removed there from by passing over the guide wire and out of the open proximal end of the sheath, concurrently to the guide wire remaining on the interior of the heart. Thereafter, the aforementioned dilator passes telescopically over and along the length of the guide wire and within the interior of the sheath through the open proximal end thereof. Further, the dilator includes a dilating tip or end portion, preferably having a substantially conical shape, with an at least minimally pointed extremity. The dilating tip passes through and forces the expansion of the entry site initially formed by the penetrating needle.

Once the entry site has been dilated the guide wire is removed from the heart through the interior of the dilator and the aforementioned stabilizer passes telescopically over the exterior of the dilator and is moved there with and/or relative thereto. The manipulation of the stabilizer assembly and more specifically the stabilizer structure relative to the dilator serve to position the stabilizer structure within the expanded entry site. The aforementioned retaining assembly, including the interior and exterior retaining members, is activated into the retaining orientations, so as to removably secure the stabilizer structure within the expanded entry site. The dilator is then removed through the interior of the stabilizer the catheter leaving the stabilizer catheter and the central lumen thereof in a position to define a free passage for the intended medical instrumentation to pass through central passage of the stabilizer structure and the entry site into the interior of the heart.

As will be explained in greater detail hereinafter, once the medical procedure intended to be performed on the interior of the heart has concluded, the entry site is closed preferably, but not necessarily, utilizing a closure assembly of the type disclosed in currently U.S. patent application Ser. No. 14/065,613, to the inventor herein.

One of the distinct advantages of the present invention is the ability to concurrently insert multiple catheters into the targeted atrium so as to enable the interaction between the concurrently present instruments within the selected atrium. Accordingly, one or more preferred embodiments of the method of the present invention comprises forming a plurality of different entry sites into the targeted atrium and corresponding pericardial wall and positioning different introductory sheaths through the correspondingly positioned ones of a plurality of entry sites. In addition, the corresponding distal ends of the plurality of the introductory sheaths have appropriate intracaridac lengths so as to facilitate the maneuverability and manipulation of the instrumentation once present in the targeted atrium. Upon completion of the required cardiac maneuvers within the selected atrium, a plurality of closure assemblies will pass through different ones of the plurality of introductory sheaths so as to operatively dispose the first and second segments of each of the closure assemblies in closing relation to the formed entry sites, as set forth above.

Therefore, the various embodiments of the present invention overcomes the disadvantages and problems associated with known surgical techniques by implementing the subject introductory assembly and method for the insertion of instrumentation through a thoracic passage into a selected portion of the heart including, but not limited to, the right or left atrium, as will be described in greater detail hereinafter.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic representation of the heart including the implementation of the present invention including the introduction of a plurality of medical instruments into a selected one of the right or left atrium of the heart.

FIG. 2A is a schematic representation of the anatomy of the heart as seen from the right chest.

FIG. 2B is a schematic representation of the anatomy of the heart as seen from the right chest and including schematic designations of surgical sites for cardiac maneuvers using the introductory assembly and method of the present invention.

FIG. 3 is a front view in partial cutaway of one preferred embodiment of the introductory assembly of the present invention.

FIG. 4 is a front view of the embodiment of FIG. 3 representing a successive step in the method of implementing the introductory assembly of the present invention.

FIG. 4A is yet another preferred embodiment of the present invention structured to be used with the introductory assembly as represented in the embodiment of FIG. 4.

FIG. 5 is another preferred embodiment of the introductory assembly of the present invention similar to but distinguishable from the embodiment of FIGS. 3 and 4.

FIG. 6 is a front view of the embodiment of FIG. 5 in a successive step of the method of implementing the introductory assembly of the present invention.

FIG. 6A is yet another preferred embodiment of the present invention structurally and operatively similar to the embodiment of FIG. 4A but modified for use in combination with at least the embodiment of FIG. 6.

FIG. 7A an exterior perspective view of the embodiment of FIGS. 5 and 6.

FIG. 7B is an end view of the embodiment of FIG. 7A.

FIG. 8A is a front perspective view of yet another preferred embodiment similar to but distinguishable from the embodiment of FIGS. 7A and 7B.

FIG. 8B is an end view of the embodiment of FIG. 8A in partial phantom.

FIG. 9 is front view in partial cutaway of the method of implementing the introductory assembly of the embodiment of FIGS. 5 and 6.

FIG. 10 is a front view of the representing the method of implementing the introductory assembly of the embodiment of FIG. 9.

FIG. 11 is a front view representing an additional step of the method of implementing the introductory assembly of the embodiment of FIGS. 9 and 10.

FIG. 12 is a composite view in perspective of a plurality of operative components comprising yet another preferred embodiment of the introduction assembly of the present invention.

FIG. 13 is a perspective view of a sheath of the introduction assembly of the embodiment of FIG. 12 extending along a thoracic passage in the chest cavity of the patient.

FIG. 14 is a perspective view of an open distal end of the sheath of the embodiment of FIGS. 12 and 13 disposed in substantially flush, confronting relation to the exterior wall of the heart.

FIG. 15 is a perspective view of an exterior of the heart and a penetrating needle of the introduction assembly of the embodiment of FIGS. 12 through 14 entering the wall of the heart.

FIG. 15A is a perspective view of the interior of the heart representing the introduction of the penetrating needle and the passage of a guide wire there through into the interior of the heart.

FIG. 16 is an exterior view of the heart and a dilator passing through an entry site in the heart into the interior thereof.

FIG. 16A is an interior view of the heart wherein a dilator of the embodiment of FIGS. 12 and 16, including a dilating tip, being guided through the entry site into the interior the heart over a guide wire.

FIG. 17 is a perspective view of the heart exterior and the at least partial activation of a stabilizer structure associated with a stabilizer assembly of FIG. 12.

FIG. 17A is a perspective view of the heart interior with a further activation of the stabilizer assembly.

FIGS. 18 and 18A are perspective views respectively on the exterior and interior of the heart wherein the stabilizer structure is fully activated in retaining relation to the dilated entry site subsequent to the removal of the dilator.

FIG. 19 is a perspective view of the heart interior representing the disposition and activation of a closure structure.

FIG. 20 is at least a portion of the retaining structure remaining activated while the closure structure of the embodiment of FIG. 19 is being applied.

FIG. 21 is a perspective view of the heart interior with an interior portion of the closure structure operatively positioned in a closed orientation.

FIG. 22 is a perspective view of the heart exterior subsequent to removal of the stabilizer assembly and the concurrent disposition of an exterior portion of the closure structure being disposed in an operative, closed orientation.

Like reference numerals refer to like parts throughout the several views of the drawings.

### DETAILED DESCRIPTION OF THE PREFERRED EMBODIMENT

As represented in the accompanying Figures, the present invention is directed to an introduction assembly and attendant method for the insertion of medical instruments, such as catheters, through a thoracic passage and corresponding intercostal spaces into either a right or left atrium of the heart for the purpose of performing predetermined cardiac maneuvers on intracardiac structures, as required.

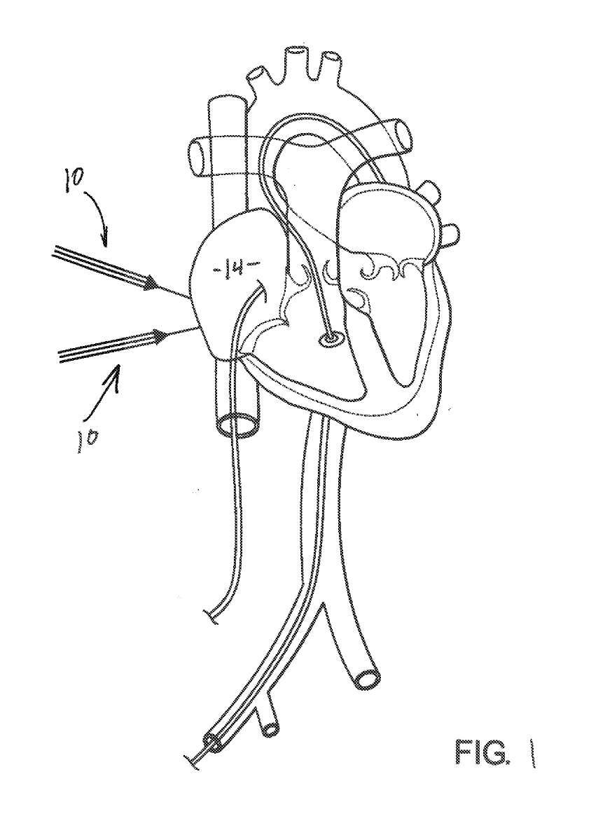

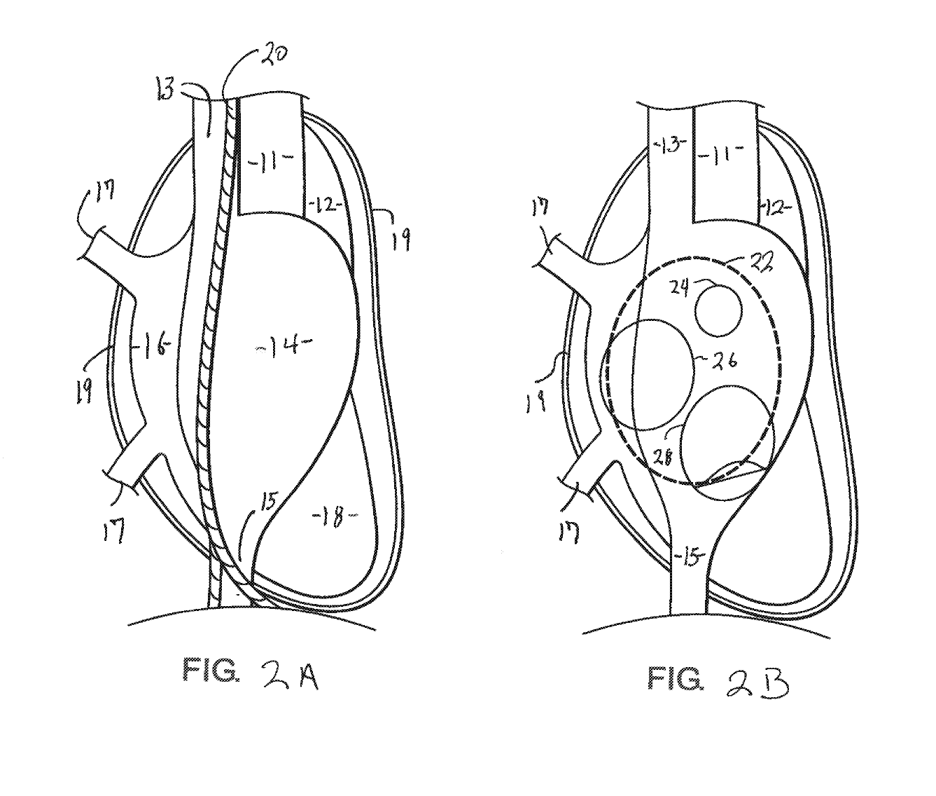

For purposes of clarity and reference, FIGS. 1, 2A and 2B are schematic representation of the anatomy of the heart. Accordingly, implementing one or more preferred embodiments of the present invention, multiple instruments, including catheters generally indicated as 10, may be concurrently disposed in either the right or left atrium of the heart. As will be set forth in greater detail hereinafter, the instruments 10 pass through the thoracic wall and appropriate ones of intercostal spaces into an interior of a targeted one of the left or right atrium by means of a formed entry site in the pericardium and selected atrium wall. In addition, FIG. 1 presents known or substantially conventional surgical techniques in which catheters are introduced into blood vessels at remote or peripheral sites from the heart and are steered through veins or arteries of the body to reach the heart.

By way of example, the femoral vessels, radial artery, subclavian artery and the jugular veins can be used for the insertion of catheters for remote cardiac intervention. As is well recognized, this peripheral approach avoids many of the risks of open heart surgery but it suffers from significant technical limitations at least partially based on the anatomy and size of the peripheral vessels or a condition existing in some patients resulting in the narrowing or calcification or torturous configuration thereof, making access to the heart difficult, as generally set forth above.

With primary reference to FIGS. 2A and 2B schematic representations of the anatomy of the heart, as seen when viewing the right chest, includes the aorta 11, pulmonary artery 12, superior vena cava 13, right atrium 14 and inferior vena cava 15. Additional representations include the pulmonary veins 17 as well as the right ventricle 18, the pericardial bag 19 and the pulmonary veins 20. For purposes of further reference, FIG. 2B provides a schematic representation of the various surgical sites in which possible cardiac maneuvers may be performed using the assembly and method of the present invention. More, specifically, the perimeter 22 generally defines the zone or area wherein multiple instruments may be concurrently introduced into the right atrium 14 through different thoracic passages and corresponding entry sites by implementing the various preferred embodiments of the present invention. Additional schematic representations include the projection of the left atrial appendage 24; the projection of the mitral valve annulus 26 and the projection of the tricuspid valve annulus 28.

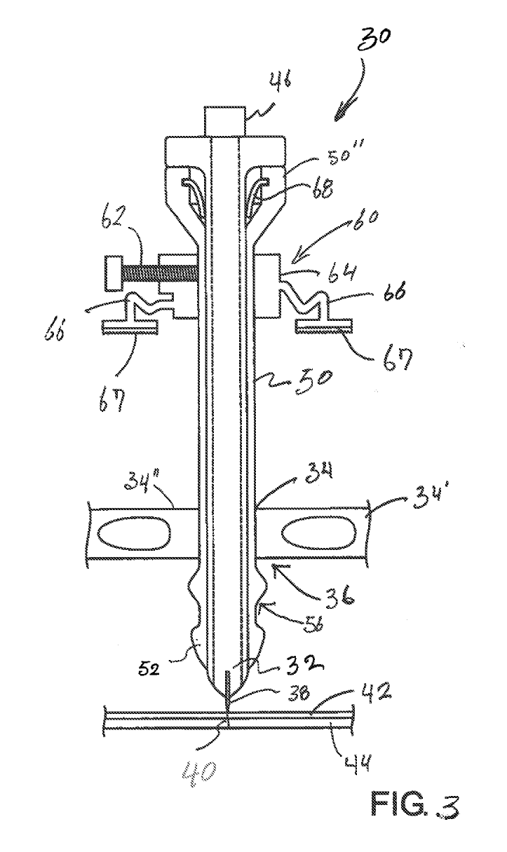

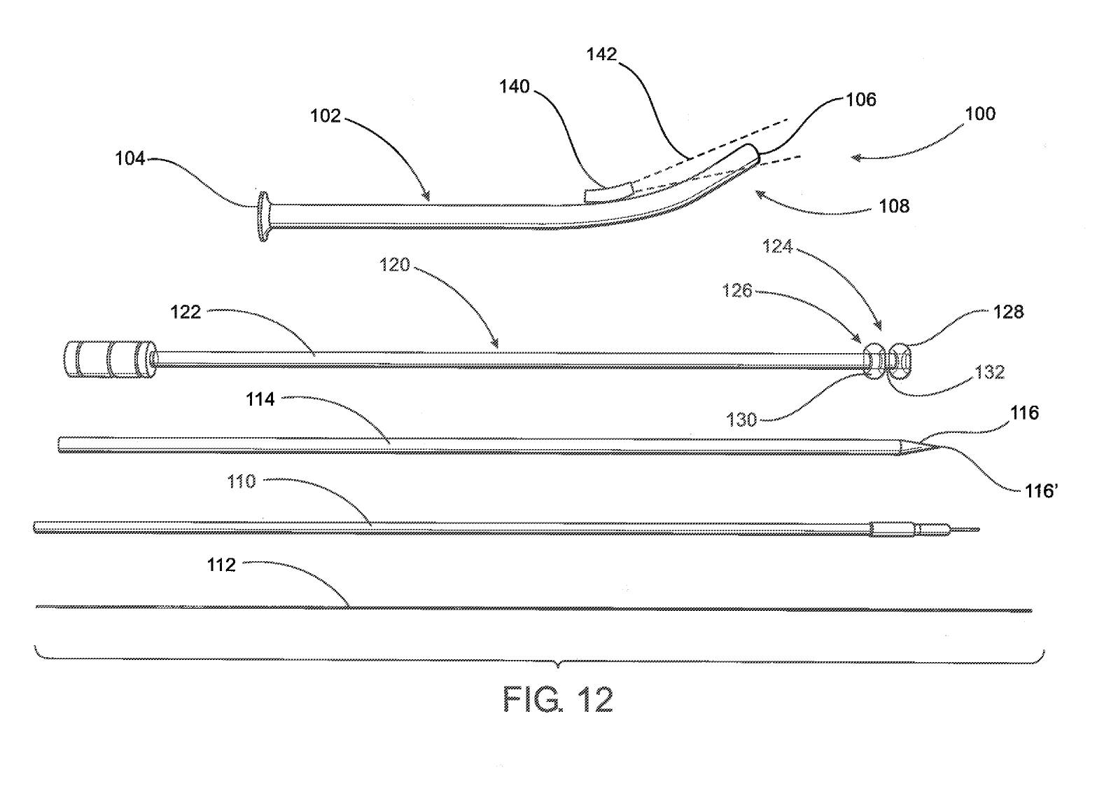

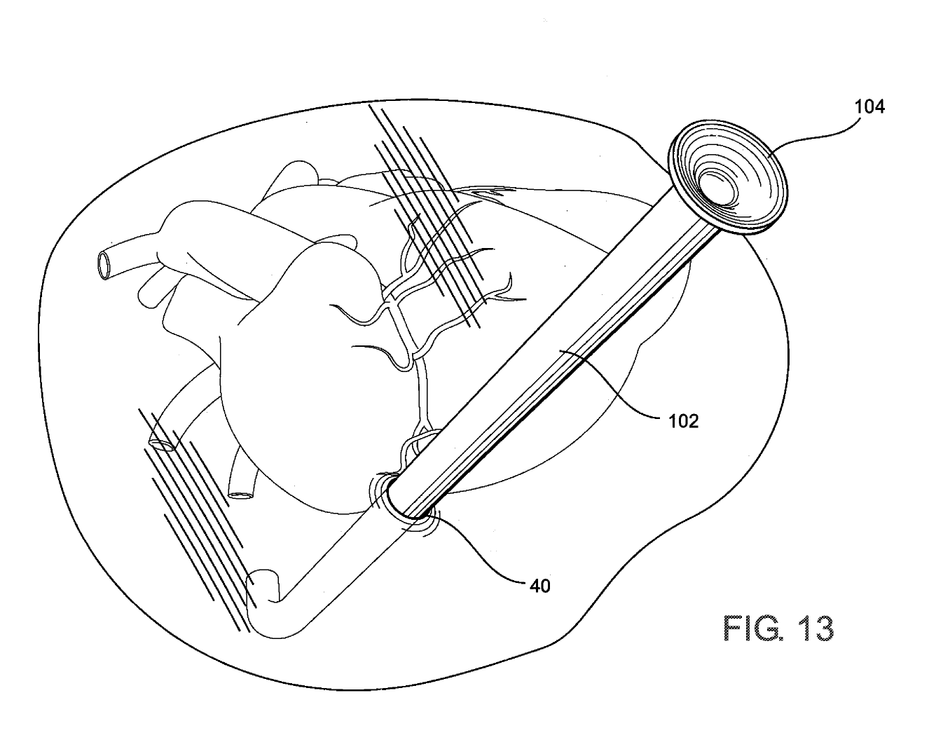

Therefore, required cardiac maneuvering of multiple catheters and other instruments can be individually and cooperatively maneuvered in the indicated surgical sites or zones by implementing the assembly and method, as described in greater detail with reference to the embodiments of FIGS. 3 through 11 and also with reference to an additional embodiment as represented in FIGS. 12 through 22.

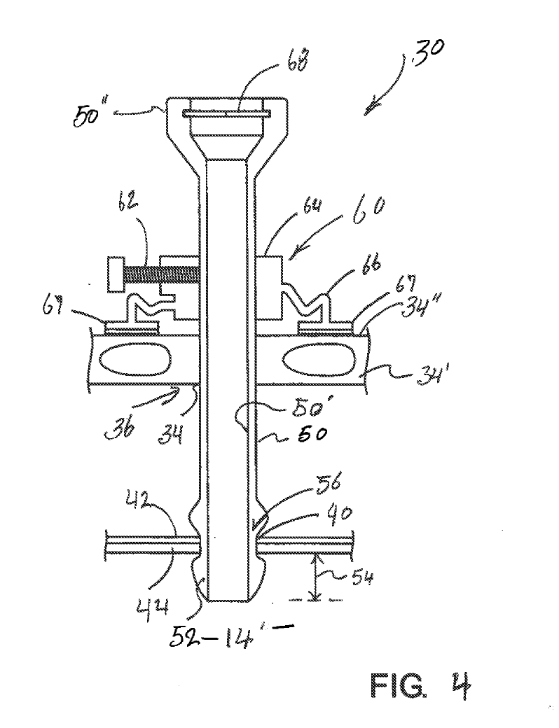

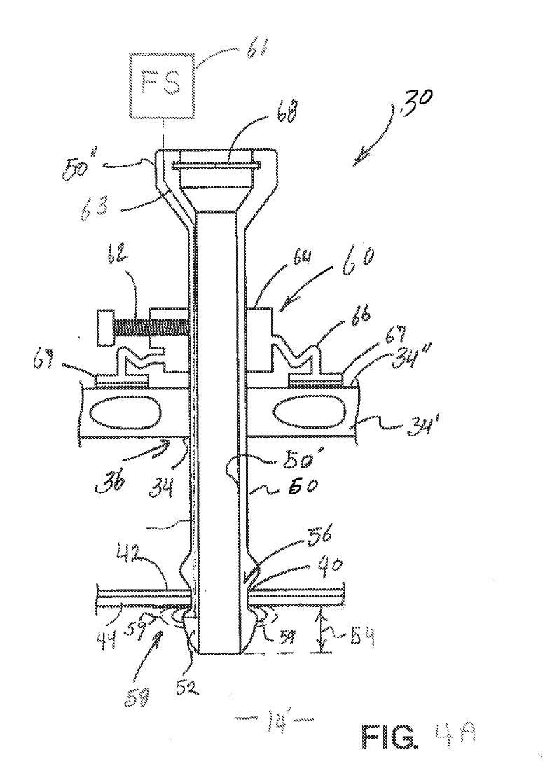

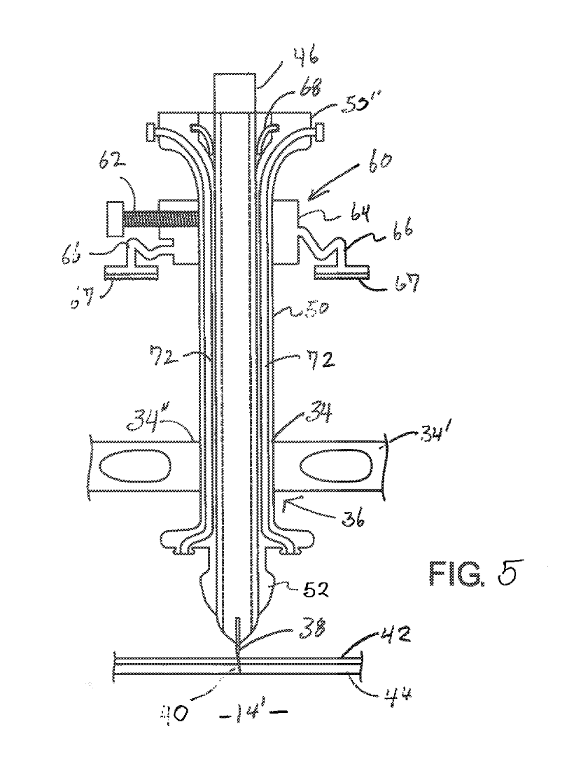

With initial reference to FIGS. 3 and 4, one preferred embodiment of the introductory assembly is generally indicated as 30. More specifically, a puncturing instrument 32, which may be in the form of a puncturing needle, lancet, etc. is utilized to form a thoracic passage 34 in the thoracic wall 34′ through an intercostal space 36 between appropriately positioned ribs, as schematically represented. Further, the lancet 32 may have a puncturing or cutting blade 38 of sufficient structure to form an entry site 40 extending through both the wall 42 of the pericardial bag and the corresponding disposed part of the wall 44 of the selected or targeted atrium 14.

While the puncturing instrument or lancet 32 may vary in construction and operation, one embodiment thereof includes the cutting blade 38 selectively disposable between an outwardly extended, operative position, as represented in FIG. 3, or an inwardly disposed retracted position, not shown for purposes or clarity. In order to accomplish this selective positioning of the blade 38, an accessible positioning member or like structure 46 is connected to the blade 38 and may be mounted on the lancet 32 at generally a proximate end thereof. As such, the positioning member 46 is disposed exteriorly of the thoracic wall 34′ and is thereby readily accessible for manipulation by medical personnel to accomplish the extension or retraction of the blade 38, as required. With further reference to FIGS. 3 and 4, an introductory sheath 50 includes a central channel or lumen 50′ facilitating the coaxial alignment and overlying, covering relation of the sheath 50 relative to the puncturing instrument 32. Once the entry site 40 is formed, the distal end 52 of the introductory sheath 50 passes there through. As a result, the central lumen 50′ of the introductory sheath 50 is disposed in accessible communication with the interior 14′ of the selected atrium 14, as generally indicated in FIG. 4.

The passage and positioning of the distal end 52 of the sheath 50 is controlled and/or restricted through the provision of a buffer, generally indicated as 56. As will be apparent from additional description provided hereinafter, the buffer 56 may be defined by a variety of different structures. However, in each of the possible structural modifications, the buffer 56 is disposed and configured to limit or restrict, and therefore at least partially define or determine, the length of the distal end 52 which passes into the interior 14′ of the selected or targeted atrium. More specifically, the disposition and structural features of the buffer 56 will determine an “intracardiac length” 54 of distal end 52 which defines the length of the distal end 52 allowed to be inserted within the interior 14′ of the selected atrium. While the intracardiac length 54 may vary, the conventional length would be generally from about 1.5 cm to 2 cm. The intracardiac length 54 is sufficient to facilitate entry of intended instruments into the atrium but is at least partially restricted to facilitate manipulation and maneuvering of a catheter or other instrument passing through the introductory sheath 50 into the interior 14′ of the targeted atrium. As a result, required cardiac manipulation of intracardiac structure intended for treatment, repair, replacement, etc. may be more efficiently accomplished.

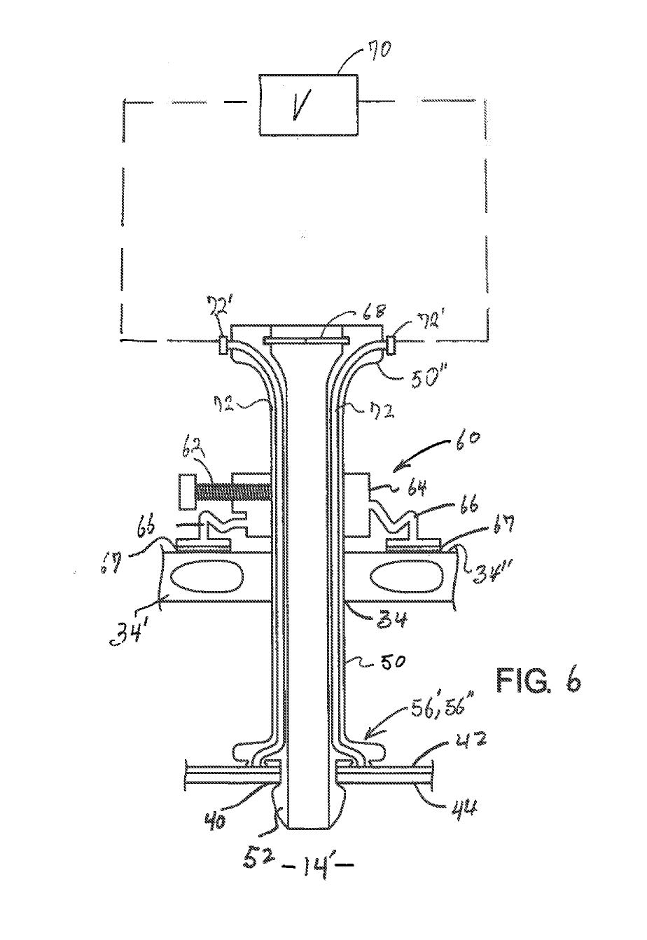

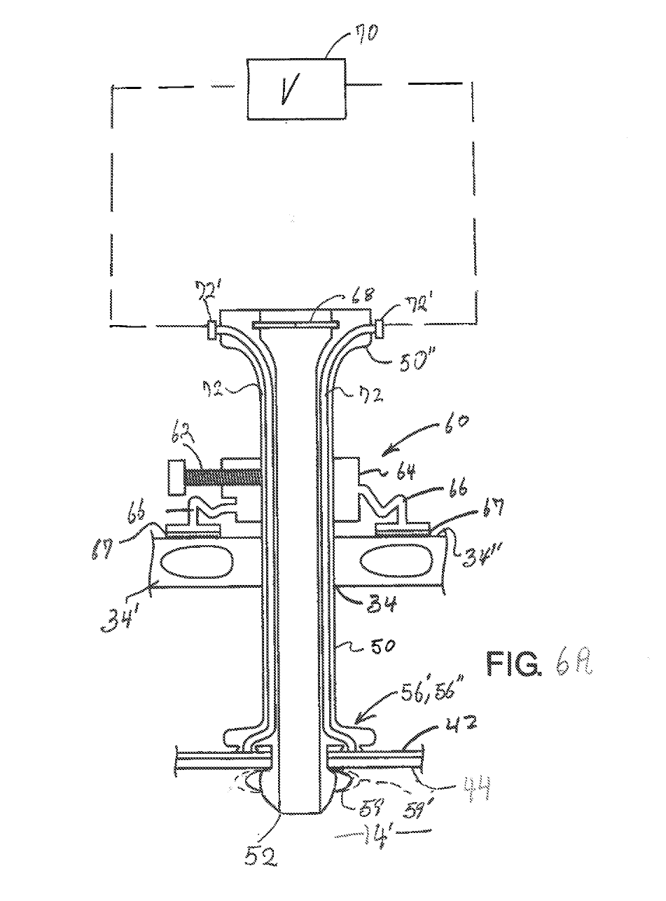

Yet another embodiment of the present invention is represented in FIGS. 4A and 6A. More specifically, the present invention further comprises a restricting assembly generally indicated as 58 which is attached to the distal end 52 of the sheet 50. Moreover, the restricting assembly 58 is preferably in the form of an expandable or inflatable bladder 59 connected to the distal end 52 as represented. Further, being expandable or inflatable, the bladder 59 is capable of being selectively disposed into a collapsed position as schematically represented in solid lines in FIGS. 4A and 6A. However, the bladder 59 is also selectively inflated or expanded as at 59′ schematically represented in phantom lines in FIGS. 4A and 6A. Accordingly, while in the collapsed position the restricting assembly or bladder 59 assumes a dimension and/or configuration which facilitates its passage through the entry site 40 as it moves with the distal end and passes through the entry site 40 into or out of the interior of the targeted atrium as at 14′. Once within the interior of the targeted atrium, the expandable or inflatable bladder may be activated to assume its restricting orientation 59′. Such an expansion or inflation may be accomplished by the provision of a pressurized fluid source 61 connected in fluid communication, as at 63, of FIG. 4A with the restricting assembly 58 and bladder 59.

Accordingly, once in the restricting orientation 59′ the bladder 59 assumes a dimension and/or configuration, extending outwardly from the distal end 52 sufficient to prevent or significantly restrict the removal of the distal end 52 from the interior of the targeted atrium. Accordingly, the restriction assembly 58 may be used in combination with or independent of the buffer 56, as represented in FIG. 4A or the additional embodiment 56′ and 56″ of the buffer as represented in FIG. 6A. It should be apparent that the restricting assembly 58 may also facilitate maintenance of the intracardiac spacing 54 of the distal end 52 when disposed within the interior of the targeted atrium 14 as represented in both FIGS. 4A and 6A. For purposes of clarity, the pressurized fluid source 61 and the fluid communicating connection 63 is absent from the representation of FIG. 6A.

Additional structural and operative features of the introductory assembly 30 include a stabilizing assembly 60 adjustably and/or movably connected to the introductory sheath 50. The stabilizing assembly 60 is selectively positioned relative to the exterior of sheath 50 into and or out of engagement with the exterior surface 34″ of the thoracic wall 34′. Moreover, the structural and operative features of the stabilizing assembly are such as to maintain a preferred and/or predetermined angular orientation of the sheath 50 relative to the thoracic wall 34′ as the sheath 50 passes through the thoracic passage 34 and the entry site 40. While the schematic representations of FIGS. 4-6 and 9-11 show a substantially perpendicular or direct inline relation between the axis of the sheath 50 and the thoracic wall 34′, FIG. 1 more accurately indicates that the various instruments 10 may assume a variety of different angles as they extend through the thoracic wall into the selected atrium. Therefore, the stabilizing assembly 60 includes a lock or like fixing member 62 movable relative to a base 64 into a removable locking engagement with the exterior of the introductory sheath 50. In addition, adjustable legs or like members 66 have engaging pads 67 structured to resist or restrict relative movement between the exterior of the pad 67 and the exterior surface 34″ or the thoracic wall 34′ to which the stabilizing assembly 60 is removably secured. As a result, the stabilizing assembly 60 facilitates the maintenance of the sheath 50 and instruments passing there through at a preferred predetermined angular orientation relative to the thoracic wall 34′.

As also indicated in one or more of the various preferred embodiments of introductory sheath 50, a valve structure generally indicated as 68 is connected at or adjacent to the proximal end 50″. More specifically, the valve structure 68 is disposed within a portion of the interior lumen 50′ and is structured to facilitate the passage of instruments into and through the lumen 50′ as they are introduced into the open proximal end 50″, as clearly represented in FIGS. 5 and 9 through 11. However, the valve structure 68 will automatically close absent the existence of instrumentation within the interior lumen. In its closed orientation, as represented in FIGS. 4 and 6, the valve structure is operatively disposed to prevent back bleeding and/or air embolism and while enabling the sequential introduction of dedicated catheters to perform the intracardiac maneuvers.

Therefore, the valve structure 68 may be considered, but is not limited to, a one way valve structure which may include an inherent bias or other operative structure which facilitates its closure into fluid sealing relation to the interior lumen 50′ absent the presence of instrumentation within the lumen 50′.

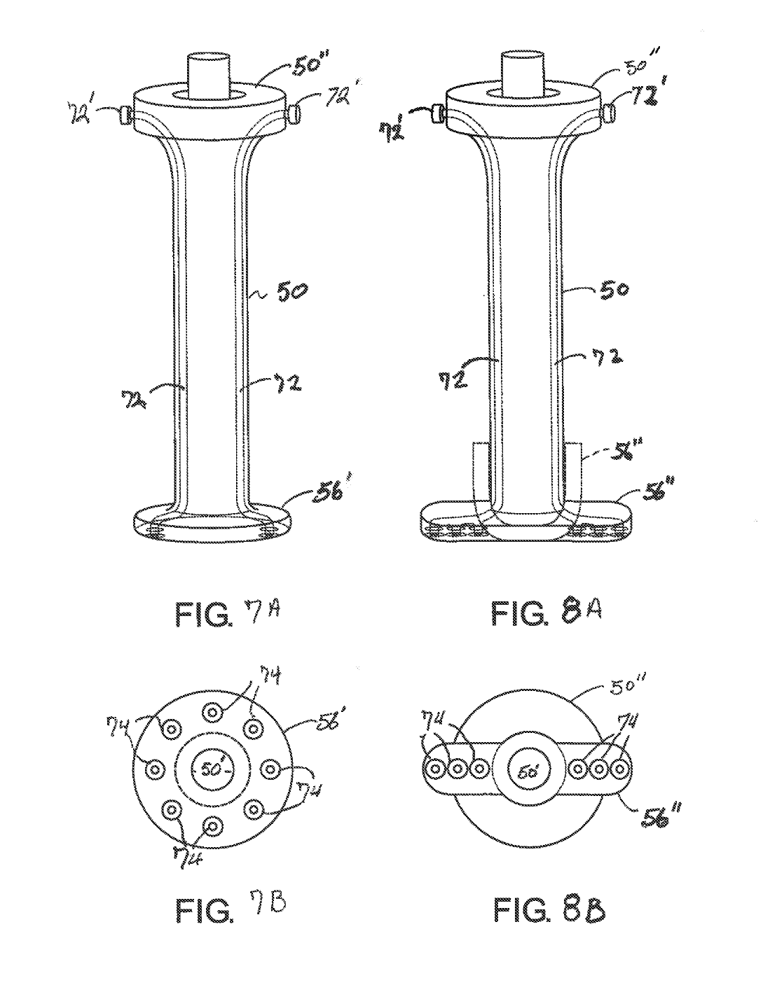

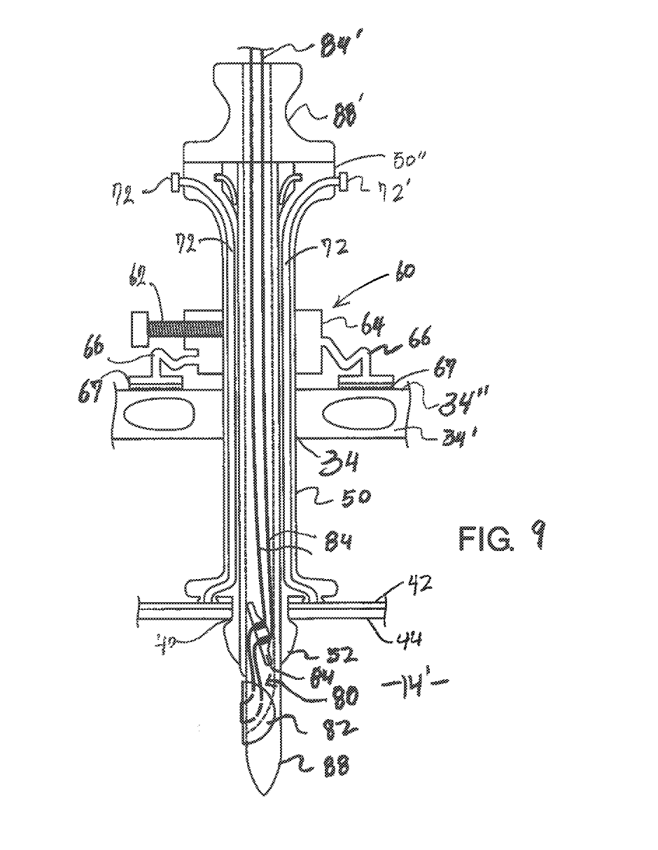

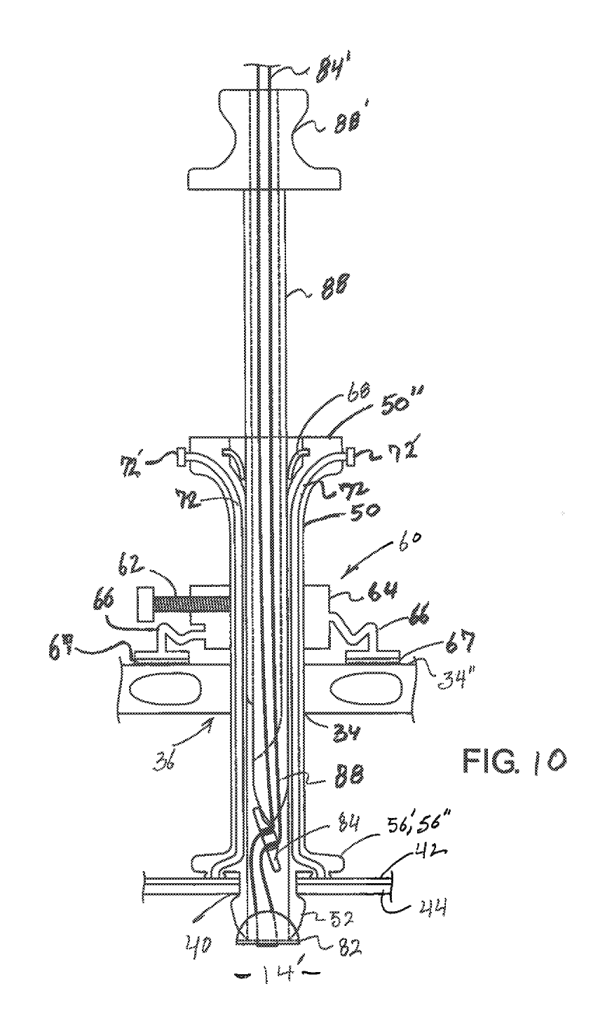

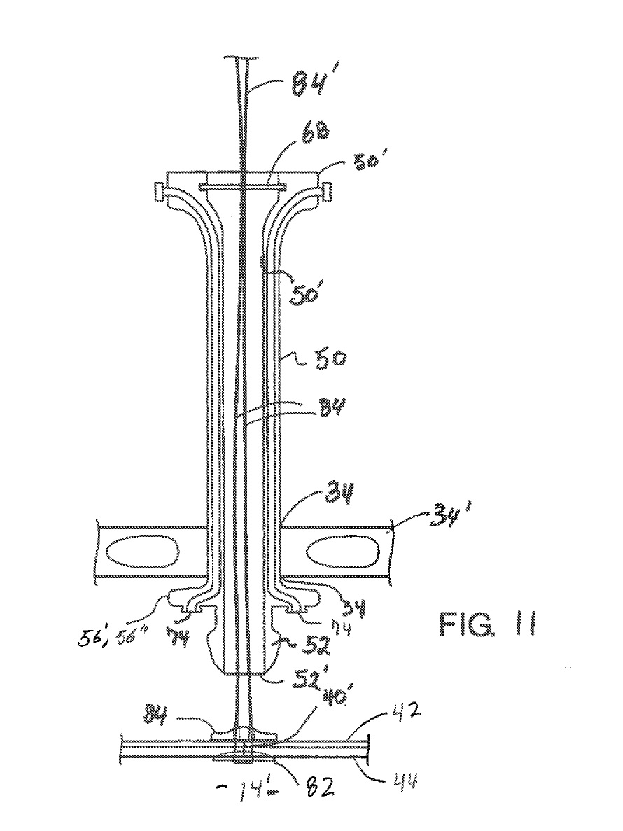

As represented in FIGS. 5 through 11 yet another preferred embodiment of the present invention comprises structural modifications of the buffers, generally indicated as 56′ and 56″. The structural and operative differences are described in greater detail with primary regard to FIGS. 7A, 7B, and 8A, 8B. More specifically, each of the buffers 56′ and 56″ is secured to the exterior of the pericardial bag 42 by means of vacuum or negative pressure generated by a vacuum source generally indicated as 70. The vacuum source 70 is connected in fluid communication to the buffers 56′, 56″ by means of appropriate conduits 72 or other interconnecting flow communicating structure. As such, the flow communicating structures or conduits 72 may be mounted on or at partially within the introductory sheath 50. As selectively operated, the vacuum source 70 may produce a negative pressure on or with the buffer structure 56′, 56″ which in turn is exerted on the exterior surface of the pericardial bag 42. As a result, the buffers 56′ and 56″ will be maintained in a secure, stable but removable engagement with the exterior of the pericardial bag 42. Such a removable securement will further facilitate the stable, intended positioning of the distal end 52 within the interior 14′ of the targeted atrium.

As should be apparent, control or regulation of the negative pressure exerted by the buffer 56′, 56″ on the pericardium 42 may be regulated by the operation of the vacuum source 70. Therefore, when activated sufficient negative pressure is exerted on the exterior surface of the pericardium 42 by the buffer 56′, 56″ in order to maintain the buffer 56′, 56″ in secure engagement therewith. However, by diminishing or eliminating the negative pressure, by regulating the operation of the vacuum source 70, a detachment of the buffer 56′, 56″ as well as the introductory sheath 50 from the entry site 40, as represented in FIG. 11, can be easily accomplished. Additional structural features associated with FIGS. 6 through 11 include the vacuum or negative pressure source 70 being removably connected to the proximal end 50″ of the introductory sheath 50 by appropriate connectors 72′ attached to or associated with the fluid flow conduits 72.

With primary reference to the embodiment of FIGS. 7A and 7B, the buffer 56′ comprises a substantially annular configuration including at least one but more practically a plurality of openings 74 formed in the under surface thereof. As should be apparent, the openings 74 are disposed in direct fluid communication with the exterior surface of the corresponding pericardial bag 42 as represented in FIGS. 6 and 9-10 and thereby exert the aforementioned negative pressure on the outside or exterior surface of the pericardial bag 42. As set forth above, the negative pressure is sufficient to maintain a secure engagement of the buffer 56′ with the exterior surface of the pericardial bag 42 thereby maintaining the stability and accurate disposition of the introductory sheath 50.

With primary reference to FIGS. 8A and 8B, yet another embodiment of the buffer 56″ is represented which includes at least one but preferably a plurality of outwardly extending pads 57. Each of the pads 57 is disposed in fluid communication with the vacuum source 70 through the aforementioned conduits or like flow communicating structures 72. Somewhat similar to the embodiment of FIGS. 7A and 7B, the pads 57, defining the buffer 56″, also include a plurality of opening 74 which are disposed in confronting engagement of the exterior surface of the pericardium 42 and thereby exert a suction or negative pressure thereon. The exerted negative pressure is sufficient to maintain the buffer 56″ into a stable but removable connection with the pericardial bag 42 substantially adjacent the entry site 40. Additional structural features of the buffer 56″ include its ability to be selectively disposed in a collapsed or retracted orientation as represented in phantom lines in FIG. 8A. As should be apparent, when in the collapsed position, the pads 57 of the buffer 56″ take up less room thereby facilitating the positioning thereof into the intended operative position as they are disposed through the thoracic passage 34 of the thoracic wall 34′.

Further, the positioning or orientation of the pads 57 in the operative position may be at least partially “automatic” by structuring the pads from a material which has at least a minimal inherent bias. Once the buffer 56″ is disposed in confronting and/or adjacent relation to exterior surface of the pericardium 42 the inherent bias of the material from which the pads 57 are formed will facilitate their “automatic” outward orientation into the operative position of FIGS. 8A and 8B.

Yet another embodiment of the present invention is represented in FIGS. 9 through 11 and is related to a closure assembly generally indicated as 80. However, it is emphasized, that the closure assembly 80, while specifically represented for use with the embodiments of FIGS. 5 through 11 is also operatively structured for use with the embodiments of FIGS. 3 and 4 as described above. Therefore, the closure assembly 80 is selectively disposable within the lumen 50′ of the introductory sheath 50 and for positioning in closing or sealing relation to the entry site 40 An operative positioning of the closure assembly 80 is accomplished upon a removal of the distal end 52 from the interior 14′ of the selected atrium, as represented in FIG. 11. For purposes of clarity the closed or sealed entry site is represented in FIG. 11 as 40′. Moreover, the closure assembly 80 includes a first segment 82 and a second segment 84 at least initially disposed in separated relation to one another. However, in at least one preferred embodiment of the closure assembly 80 includes an interconnecting structure, such as a cord or like structure 84, which may be manipulated interconnect the first and second segments 82 and 84 into the closing relation to the entry site 40′. As such, the interconnecting structure 84 extends through substantially the entire length of the lumen 50′ and includes a portion 84′ which is assessable from the exterior of the introductory sheath 50, as clearly indicated. As implemented, the first segment 82 passes into the interior 14′ of the selected atrium through the open entry site 40 formed in the pericardium 42 and the atrium wall 44. Such interior positioning of the first segment 82 may be accomplished by appropriate instrumentation 88 which also may be in the form of a positioning catheter or like structure. The instrumentation 88 also passes through the interior lumen 50′ of the introductory sheath 50 and includes a positioning member 88′ protruding outwardly from the open proximal end 50″ of the introductory sheath 50 as represented in FIGS. 9 through 11. With primary reference to FIG. 10, once the first segment is disposed on the interior 14′ of the selected atrium, the second segment 84 is disposed or remains within the interior lumen 50′ adjacent to the distal end 52. Once the first segment 82 is disposed on the interior 14′ of the atrium, the distal end 52 of the introductory sheath 50 is removed from the interior 14′ of the selected atrium and passes back through the open entry site 40 along with the second segment 84 remaining on the interior of the lumen 50′.

Subsequent to the removal of the distal end 52 of the introductory sheath 50 from the entry site 40 and upon closure of the entry site 40, as at 40′, the positioning instrument 88 will serve to remove the second segment 84 from the interior lumen 50′ through the opening 52′ of the distal end 52. Appropriate manipulation of the exterior, accessible end 84′ of the interconnecting structure 84 will then serve to dispose both the first segment 82 and the second segment 84 into the closing relation to the now closed entry site 40′ as clearly represented in FIG. 11. When in the operative closing relation as represented in FIG. 11, the first closing segment 82 will be disposed in confronting engagement with the interior surface of the selected or targeted atrium wall 44. In cooperation therewith, the second exterior closing segment 84 will be disposed in confronting engagement with the exterior surface of the pericardium 42. As such the closed entry site 40′ will thereby be effectively “sandwiched” therebetween to prevent leakage or passage of fluid therethrough. This closing sealing relation of the closing assembly 84, relative to the closed entry site 40′, will facilitate the healing thereof.

Additional features of the closure assembly 80 and specifically including the first and second closing segments 82 and 84 are their formation from a material which has an at least minimal inherent bias. As such, both the first and second closing segments 82 and 84 may be disposed in at least partially folded or otherwise collapsed orientation as they pass through the interior lumen 50′ of the introductory sheath 50. However, once passing out of the opening 52′ of the distal end 52, the “inherent bias” of the material of the first and second closing segments 82 and 84 will facilitate their “automatic” expansion into the operative position clearly represented in FIG. 11. Also of note is the forming of the first and second closing segments 82 and 84 from a material that will eventually dissolve on a timely basis by the exposure to ambient bodily fluids. The time in which the first and second closing segments 82 and 84 will be dissolved effectively coincides to the healing of the closed entry site 40′.

Accordingly, the introduction assembly and method for the insertion of medical instrumentation through a thoracic passage into a targeted atrium of the heart overcomes many of the disadvantages and complications associated with conventional or known related surgical procedures, as set forth above.

By implementing one or more of the embodiments of FIGS. 3 through 11, the attendant method comprises forming at least one, but if required, a plurality of entry sites 40 into a targeted atrium 14 and positioning different introductory sheaths 50 through different thoracic passages 34 and corresponding ones of the formed entry sites 40. The distal end 52 of each of the introductory sheaths 50 is inserted through corresponding entry sites 40 into the interior 14′ of the selected atrium 14 to a depth corresponding to the intracardiac length 54 of the inserted distal end 52. Once the one or more sheaths 50 are inserted through respective ones of the entry sites 40, catheters or other instruments dedicated to perform predetermined cardiac maneuvers pass through the one or more introductory sheaths 50 into the targeted atrium 14 through the corresponding entry sites 40. Thereafter and upon completion of the required cardiac maneuvers, the inserted catheters or instruments are removed from the interior 14′ of the targeted atrium 14 back through the central lumen 50′ of the respective introductory sheaths 50.

In order to close or seal the entry sites 40 a plurality of closure assemblies 80 are passed through the interior lumen 50′ of each of the one or more introductory sheaths 50. In establishing a closing relation of the closing assemblies 80 with the entry sites 40, a first closing segment 82 and a second closure segment 84 of each closure assembly 80 are respectively disposed interiorly and exteriorly of the entry site 40. As such, the entry sites 40, or 40′ when closed, are disposed in a substantially “sandwiched” relation between the corresponding first and second closure segments 82 and 84. After operative positioning of the closure assemblies 80, each of the one or more introductory sheaths 50 are removed from the operating field by movement back through the respective thoracic passages 34.

Yet additional preferred embodiments of the present invention include a portion of said sheath 50 formed of a flexible material. Moreover, the flexible material should be structured to demonstrate sufficient and/or a predetermined minimum amount of flexibility to accommodate relative movement between the pericardial bag 42 and at least the corresponding wall 44 of the targeted atrium 14 adjacent to the entry site 40. In addition, this predetermined amount of flexibility should be sufficient to eliminate or significantly reduce the possibility of tearing, ripping or like damage being done to the relatively fragile wall tissue 44 of the targeted atrium 14. Absent this sufficient flexibility in the distal end 52, as well as a length of the sheath 50 extending from the distal end and engaging, passing through and/or correspondingly disposed with the entry site 40, damage of the targeted atrium wall 44 may occur. Such damage may be the result of, but not limited to, forces placed on the atrium wall 44 and/or pericardium bag 42 by a non-flexible portion of the sheath 50 which passes through or is sufficiently close to the entry site 40 and/or which is disposed within the targeted atrium 14, when relative movement or displacement occurs between the pericardium bag 42 and the atrium wall 44.

Yet another preferred embodiment of the present invention is directed to an introduction assembly generally indicated as 100 and represented in the various stages of application in FIGS. 13 through 22. In addition, FIG. 12 comprises a composite view of the various operative, interactive components of the introduction assembly 100. The interactive components specifically include, but are not limited to, a sheath 102 preferably formed of a rigid material along all or a significant portion of its length and including an open proximal and well for an open distal end 106. As will be explained in greater detail hereinafter, the rigidity of the entire or substantial portion of the length of the sheath 102 facilitates its manipulation and disposition an operative placement relative to the exterior of the heart and an entry site 111, which is to be formed therein. The sheath 102 has a central lumen extending along the length thereof between and in communicating relation with an open proximal and distal ends 104 and 106 respectively. Further the sheath 102 is preferably made from a stainless steel or other rigid material, wherein the rigidity as well as other structural features of the sheath, serve to facilitate the manipulation and positioning thereof relative to the heart. More specifically and as represented in FIG. 13, the sheath passes through an entry site 40 in the thoracic wall and passes along a thoracic passage within the chest cavity into operative placement relative to the heart 107.

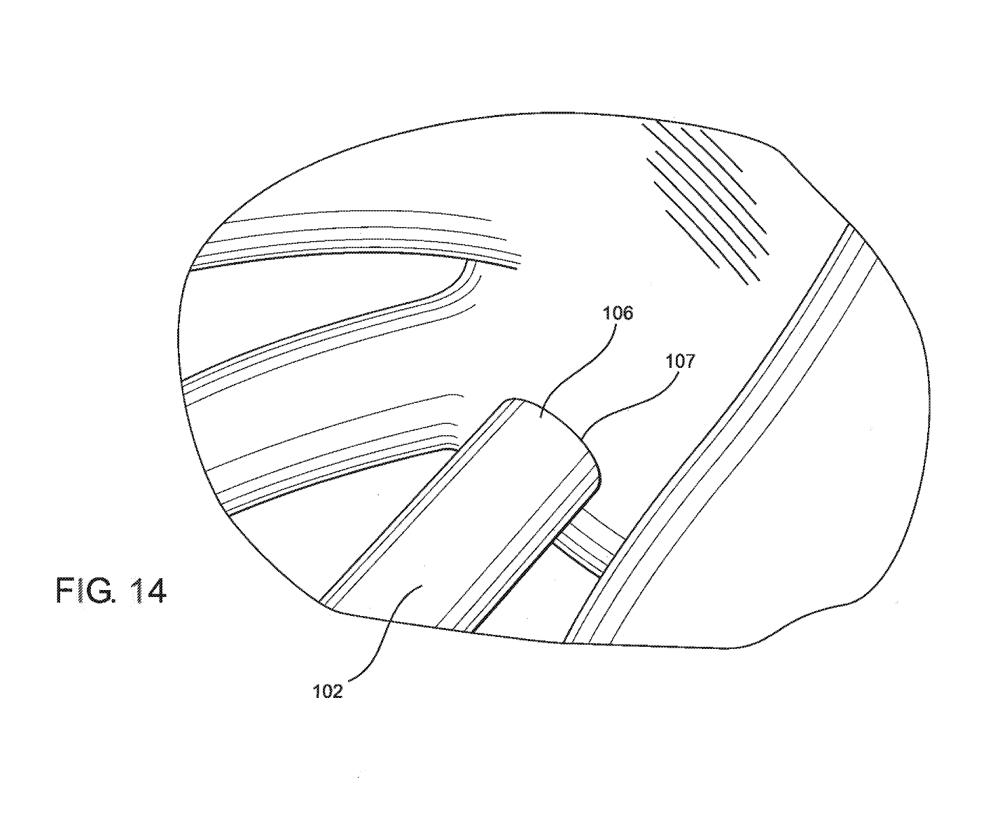

As with the additional embodiments, described above, the operative placement of the sheath, as well as the other components associated with the introduction assembly 100 may include a substantially flush, confronting relation of the open proximal end 106 relative to the exterior of the heart and in alignment with the portion of the heart associated with an intended medical procedure. However a purpose of the introduction assembly 100 is to facilitate the entry an introduction of various instruments or medical devices into the left or right atrium or other portions of the heart where medical procedures are required. Additional features of the sheath 102 further facilitate the operative placement of the open distal end 106 into substantially flush, confronting relation with the exterior of the heart wall, as clearly represented in FIG. 14. Such additional features include, but are not limited to, a predetermined length of the sheath 102, generally indicated as 108, having a predetermined configuration. As represented the length or portion 108 having the predetermined configuration is preferably formed into a curved configuration. Moreover, the curved length 108 extends generally continuously from the open distal end 106 along the sheath 102 towards, but in clearly spaced relation from, the open proximal end 104.

As explained in greater detail hereinafter, the predetermined, curved configuration 108, along with the rigidity of the sheath 102 combine to facilitate the accurate and effective positioning of the sheath 102 within the chest cavity and along the aforementioned thoracic passage and into the into the operative placement with the exterior of the heart wall. As emphasized herein, the operative placement specifically, but not exclusively, includes the substantially flush, confronting relation of the open distal end 106 with the exterior of the heart wall. As a result an entry site 111 will be formed in the heart. It is to be noted that the entry site 111 is meant to refer to and designate the entry site formed in the heart wall and is clearly distinguishable from the entry site “40” formed in the thoracic wall and referred to with regard to the embodiments of FIGS. 1 through 11.

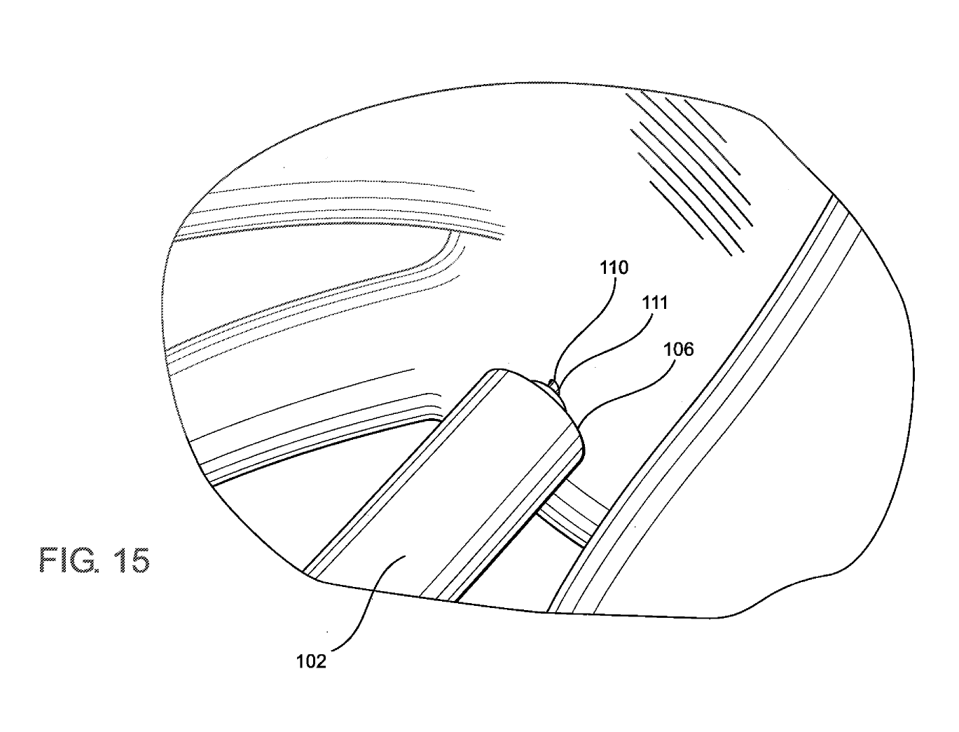

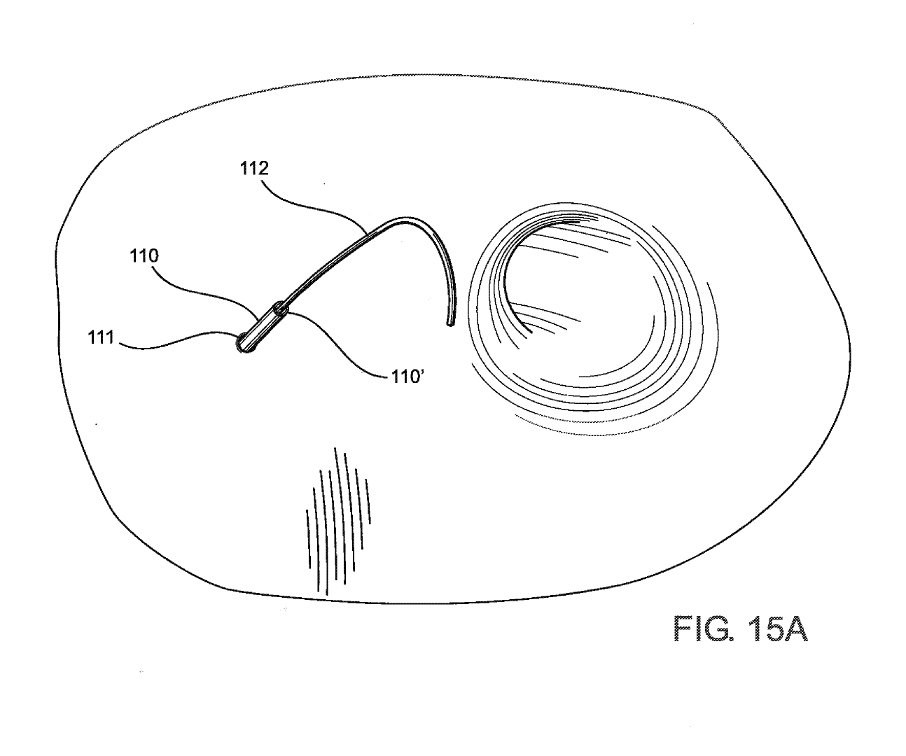

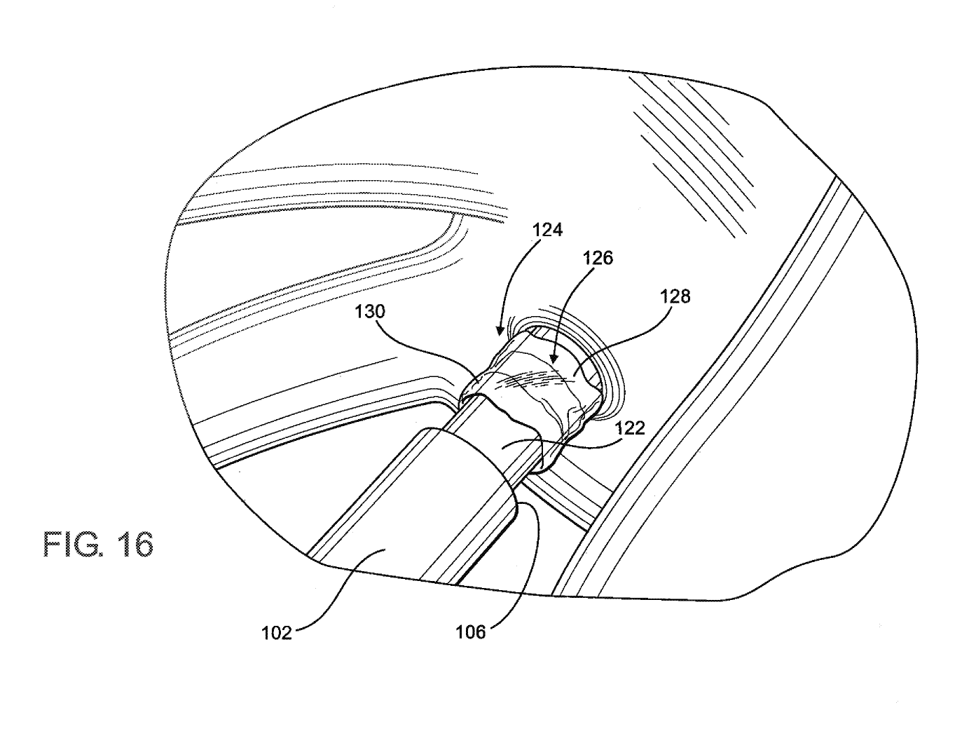

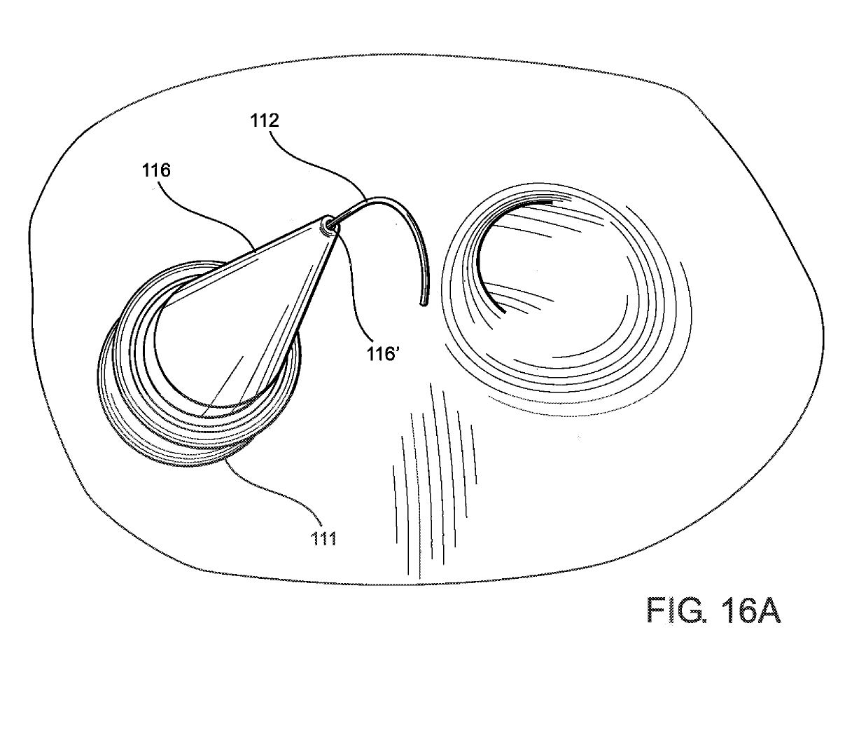

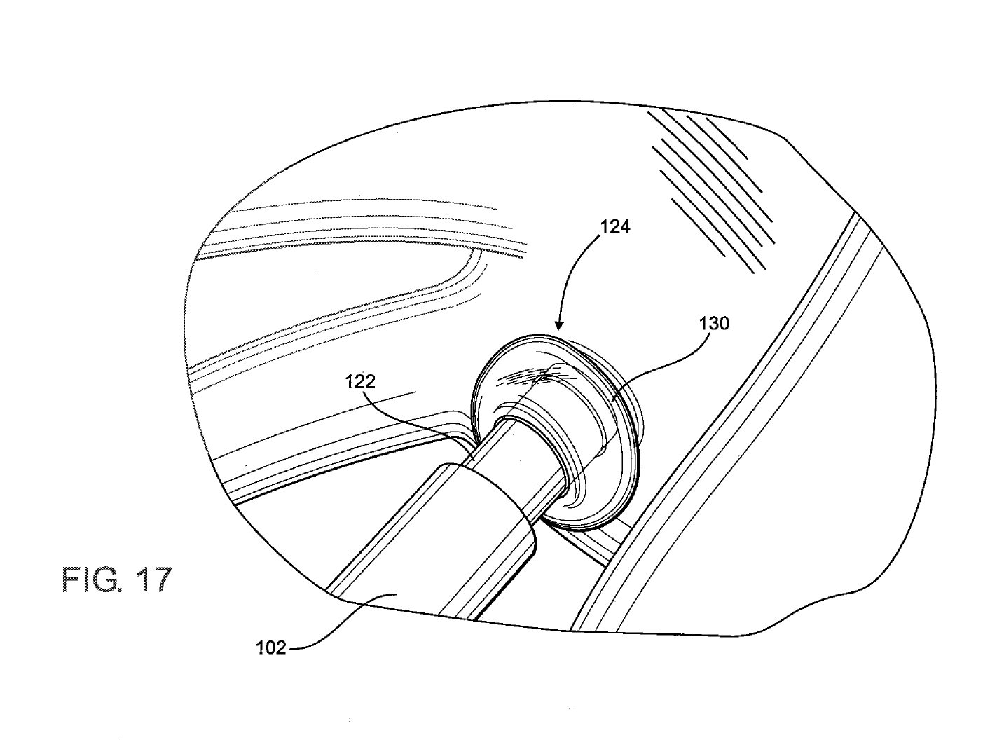

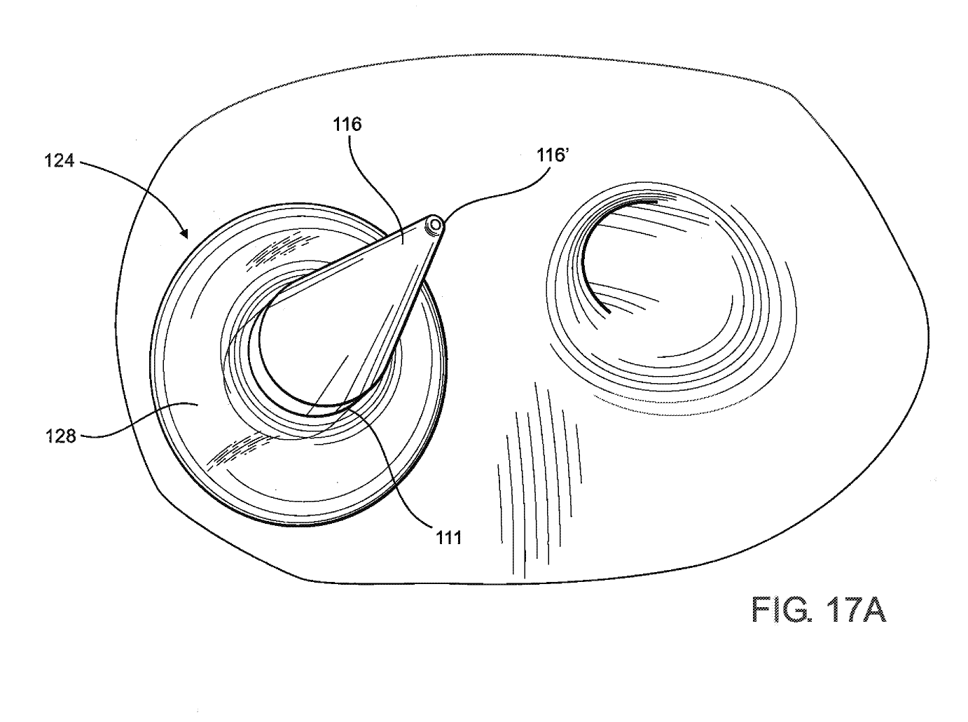

Further with primary reference to FIG. 12, additional operative and interactive components of the introduction assembly include a penetrating needle 110 and a guide wire 112. As explained in greater detail with additional reference to FIGS. 15, 15A, 16 and 16A, the penetrating needle 110 will be used to actually establish the entry site 111. As such, the penetrating needle 110 includes a hollow interior and open distal end 110′ through which the guide wire 112 passes as it is telescopically movable within the interior of the penetrating needle 110.

With further reference to FIG. 12, the introduction assembly 100 also includes a dilator 114 having an elongated configuration and a hollow interior extending along its length. Further the dilator 14 includes a distal extremity in the form of a penetrating tip 116. The penetrating tip 116 is dimensioned and configured to facilitate an expansion or dilation of the entry site 111, once it is initially established by the forced entry of the penetrating needle 110, as will be explained in greater detail with reference to FIGS. 16 and 16A. The preferred configuration of the penetrating tip 116 may be substantially or at least partially conical and have an extremity 116′ which is at least partially pointed. The extremity 116′ of the tip 116 also includes an open end communicating directly with the lumen or central passage disposed in an along the length of the dilator 114. Therefore, the dilator 114 moves telescopically over the guide wire 112 and telescopically within the interior of the sheath 102 and is accurately disposed in alignment with the entry site 111. As a result, the dilator tip 116 is precisely aligned with the entry site 111 formed by the penetrating needle 110 thereby further facilitating the forced entry of the tip 116 and dilation of the entry site 111. As also explained in greater detail hereinafter, a stabilizer assembly 120 may be telescopically mounted exteriorly of the dilator 114 and move there with relative to the heart and the entry site 111, as also represented in FIG. 16.

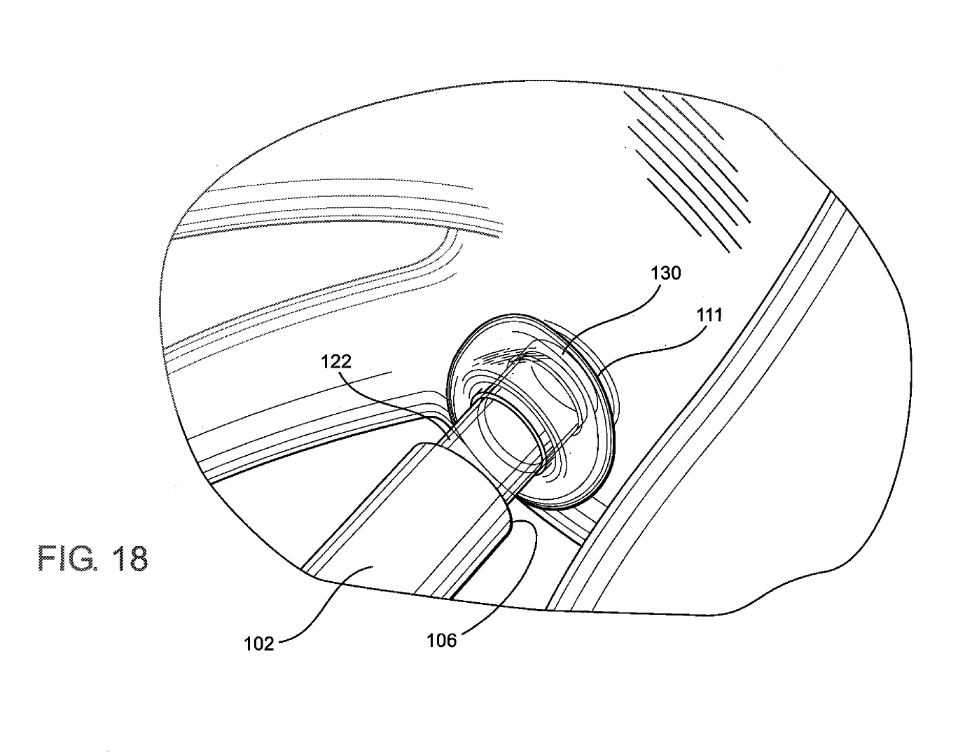

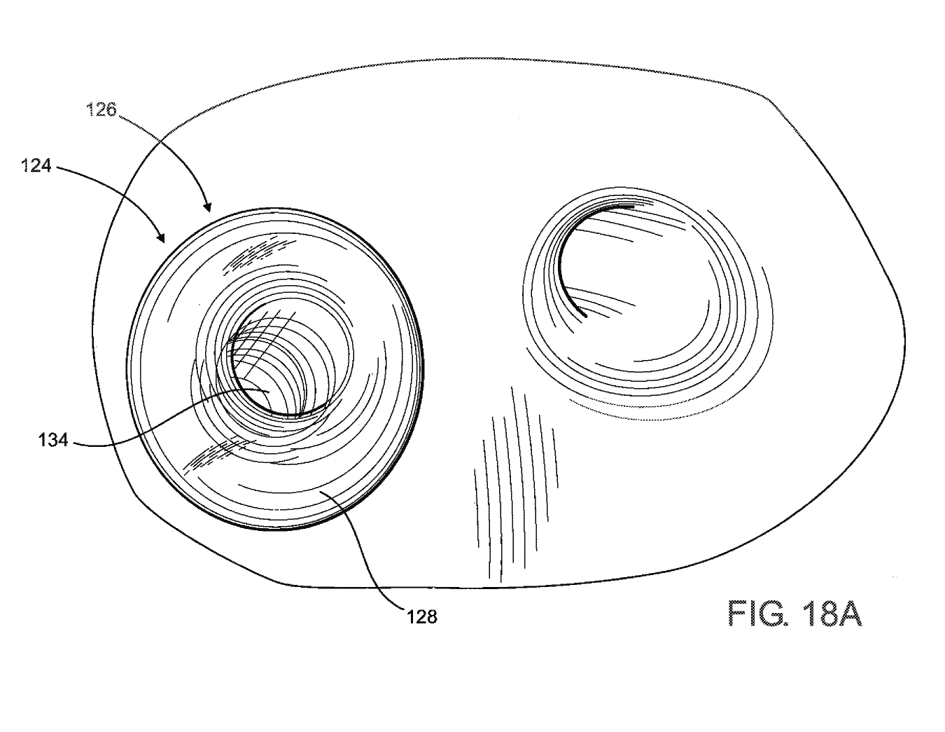

With further reference to FIG. 12, the introduction assembly 100 also includes the aforementioned stabilizer assembly generally indicated as 120. The stabilizer assembly 120 includes an elongated stabilizer catheter 122 having a central lumen or hollow interior extending along the length thereof. In addition, a stabilizer structure generally indicated as 124 is connected to the distal end of the stabilizer catheter 122. The stabilizer structure 124 is structured for removable retention within the interior of the entry site 111. As will be indicated in greater detail, the stabilizer structure 124 is removably retained within the entry site 111 so as to maintain the entry site 111 in the expanded or dilated orientation established by the forced insertion of the dilator tip 116. In addition, the stabilizer structure 124 includes a retaining assembly generally indicated as 126 comprising spaced apart retaining members 128 and 130 disposed in predetermined spaced relation to one another at opposite ends of the stabilizer structure in adjacent or contiguous relation to open ends of a central passage or channel 134. As will be explained in greater detail with primary reference to FIGS. 17, 17A, 18 and 18A, the space 132 between the retaining members 128 and 130 is disposed at and dimensioned to receive the portion of the heart wall surrounding the entry site 111. As such, when the stabilizer structure 124 and the retaining assembly 126 are in their intended, operative positions, relative to the entry site 111, the retaining members 128 and 130 may be accurately described and referred to as interior and exterior retaining members, respectively. This positional reference is indicative of the retaining member 128 located within the interior of the heart and the retaining member 130 concurrently located on the exterior of the heart.

With further regard to the structural and operative features of the retainer assembly 126, both the retaining members 128 and 130 may be independently disposed in a retaining orientation such as represented in FIGS. 17 through 18A. In at least one preferred embodiment, each of the retaining members 128 and 130 are structured to be selectively inflated and deflated and as such are connected by appropriate conduits or like structures to a fluid supply located exteriorly of the chest cavity. The inflating/deflating conduits serve to independently supply an inflating fluid into an out of the retaining members 128 and 130 so as to accomplish their retaining orientations independently of one another. Also, the fluid transmitting conduits, or similarly functional structure, which serves to inflate and/or deflate the retaining members 128 and 30, may be located within the interior portions or exterior portions of the stabilizing catheter 122 and are not shown for purposes of clarity.

Yet additional structural features associated with one or more of the operative components of the introduction assembly 100, include a buffer or bumper member 107 connected to and at least partially surrounding the outer periphery of the open distal end 106 as best represented in FIG. 14. The buffer 107 may be formed from a variety of different cushioning materials and or have various other structures which protect the exterior surface of the heart wall from a damaging engagement with the outer periphery of the open distal end 106. Such cushioning protection may be especially relevant when the open distal end 106 is disposed in its operative placement, in substantially flush, confronting relation to the outer surface of the heart wall and in surrounding and/or aligned relation to the entry site 111.

Also, in at least one additional embodiment a camera as at 140 may be connected to an exterior or other appropriate portion of the sheath 102 along a length thereof. The camera 140 is thereby dimensioned, disposed and operative to establish a field of view 142 of at least the entry site 111. The field of view 142 may also be sufficient to view an appropriate surrounding area relative to the entry site 111, as the sheath 102 and/or the remaining interactive components of the introduction assembly 100 are being used. It should be noted that the camera 140 is schematically represented and may be substantially different in size and location then that represented in FIG. 12 in order to accommodate passage through the entry site 40 in the chest cavity and along the thoracic passage.

In use during a medical procedure, the sheath 102 is introduced through anyone of a plurality of different entry sites 40 (see FIG. 1) dependent at least in part, on the portion of the heart wall to be engaged by the operative placement of the sheath 102 and open distal end 106. As indicated, the rigidity of the material from which the sheath 102 is formed as well as the predetermined configuration or curvature 108 facilitates an accurate and efficient steering and manipulation of the sheath into its operative placement, as set forth above. Moreover, the schematic representation of FIG. 13 is intended to represent the interior of the chest cavity and the thoracic passage along which the sheath 102 is disposed.

In use and after passage of the sheath 102 along the thoracic passage, the aforementioned operative placement includes the sheath 102 and more specifically the open distal end 106 disposed in substantially flush, confronting relation to the exterior wall of the heart at the intended location. As should be apparent, the intended location is at least partially dependent on the medical procedure being performed on the interior of the heart. Again, the protective buffer 107 may be connected in overlying relation to the periphery of the open distal end 106. Once in its operative placement, the sheath 102 may remain adjacent to and/or in aligned, surrounding relation to the entry site 111. As also indicated, the entry site 111 is at least initially formed by the penetrating needle 110 being forced through the wall of the heart from the exterior thereof as the penetrating needle 110 passes telescopically within the interior of the sheath 102. Once the open distal end 110′ enters into the interior of the heart, the guide wire 112 passes telescopically within the interior of the penetrating needle 110, until a portion thereof passes through the open and 110′ into the heart interior.

Thereafter and as represented in FIGS. 16 and 16A the dilator 114 passes telescopically within the interior of the sheath 102, telescopically over and relative to the guide wire 112 and out through the open distal end 106. Prior to introduction of the dilator 114, the penetrating needle 110 will be removed from the entry site 111 through the interior of the sheath 102. However, as represented, the guide wire 112 remains in place on the interior the heart and within the sheath 102 and serves as a guide for accurate placement dilator 114 relative to the entry site 111. More specifically, the dilator 114 moves telescopically over the exterior of the guide wire 112 and along the length thereof until the penetrating tip 116′ is forced through the initial entry site 111. As indicated, the configuration of the tip 116′, being substantially conical or other preferred shape, facilitates the expansion or dilation of the initially formed entry site 111. Once the dilating tip 116 is forced through the entry site 111 the guide wire 112 may be removed by it passing through the interior of the dilator 114 and out through the open proximal end 104 of the sheath 102.

As clearly represented in FIGS. 16 through 17A, the stabilizer assembly 120 including the elongated stabilizer catheter 122 is movably disposed telescopically on the exterior of the dilator 114 as the dilator 116 is forced through the initial entry site 111. Further, the stabilizer 120 is movable with and relative to the dilator 114 as the entry site 111 is expanded. However, once expanded, the stabilizer structure 124 is disposed along the exterior of the dilator 114 adjacent to the dilator tip 116 which is then located within the interior of the heart, as represented in FIGS. 16 and 16A. As should be apparent both the retaining members 128 and 130 are in a collapsed or non-inflated state so as to minimize the peripheral dimensions thereof. This facilitates the stabilizer structure 124 to pass into and at least partially through the entry site 111 along, with the forced positioning of the dilator 114 and or dilator tip 116. However, as represented in FIGS. 16A, 17 and 17A, once the dilator tip 116 is properly disposed within the interior of the heart, the exterior retaining member 130 may be activated into its retaining orientation, such as by being inflated, as clearly indicated in FIG. 17. Independently, the interior retaining member 128 is then activated and/or inflated so as to force it into its retaining orientation as indicated in FIG. 17A. For purposes of clarity the retaining orientation of the retaining members 128 and 130 may be defined by their expansion and/or inflation. Moreover, when the retaining members 128 and 130 are in their retaining orientation they are preferably and at least partially disposed in confronting relation to corresponding interior and exterior surfaces of the heart wall, as represented as also represented in FIGS. 17 through 18A.

Once both the interior retaining member 128 and the exterior retaining member 130 are in their retaining orientation, the stabilizer structure 124 will be removably secured in surrounding, receiving, retaining relation to the heart wall surrounding the entry site 111. This portion of the heart wall will then be located within the defined space 132 between the retaining members 128 and 130. Also, one feature of the retaining structure 126 is the establishment of the central passage 134 extending there through in direct communication with the interior lumen of the stabilizing catheter 122. As such, the interior lumen of the stabilizing catheter 122 and the central passage 134 will define the main path or entry route of various types of instrumentation into the interior of the heart to perform the required or intended medical procedure.

As also indicated, an additional feature of the cooperative structuring and dimensioning of the various interactive components of the introduction assembly 100 comprises a substantially enlarged interior dimension thereof. More specifically, the introduction assembly 100 and the interactive components associated therewith are configured to pass through the thoracic passage into the operative placement relative to the heart, as clearly represented in FIG. 13. Accordingly, the size and more specifically the interior dimension of at least the sheath 102, the stabilizer catheter 122 and the stabilizer structure 124 may be significantly larger than conventional medical devices of this type. In one or more preferred embodiments the interior diameter or transverse interior dimension of the sheath is generally in the range of 7.3 mm to 1.5 cm. This will allow the insertion of instrumentation including replacement valves and components associated therewith into the interior of the heart being of a size which would not be introduced through catheters reaching the heart through the blood vessels, as described above. Moreover, if the interior diameter of the stabilizer catheter 122 and stabilizer structure 124 is equal to or greater than 7.3 mm, the interior diameter of the sheath 102 must be at least sufficiently greater to allow telescopic movement of the stabilizer sheath 122 and stabilizer structure 124 to pass along the length of the sheath 102 and out through the open distal end 106.

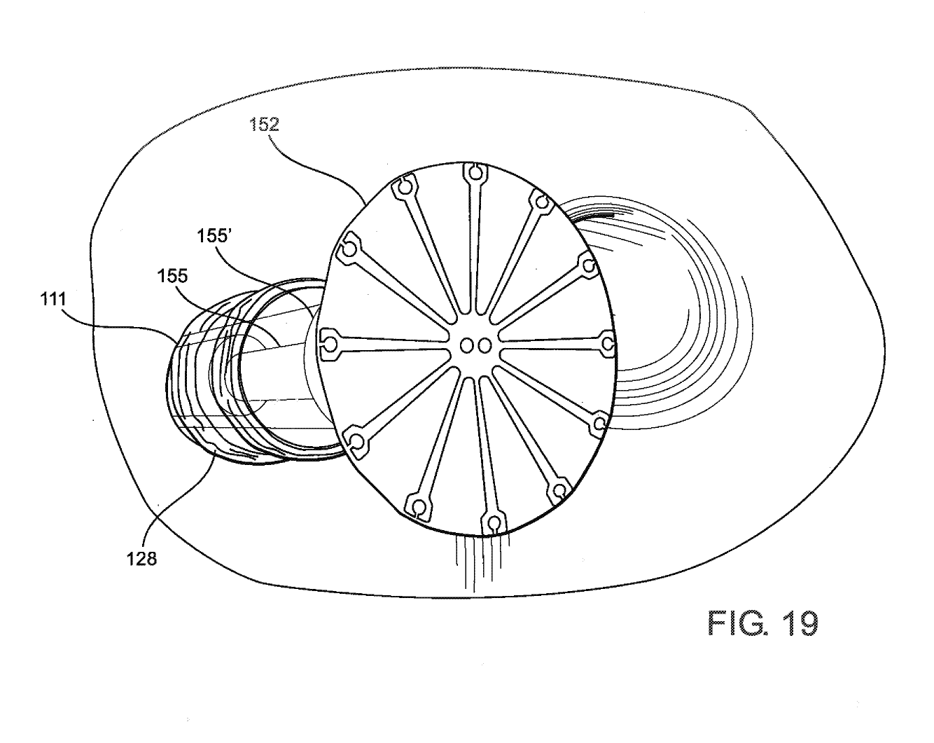

Upon completion of the surgical procedure within the interior of the heart, the entry site 111 must be closed and sealed to prevent leakage and facilitate proper healing. Accordingly as represented in FIGS. 19 through 22, a closure assembly, generally indicated as 150, may include an interior closure member 152 and an exterior closure member 154 of the type disclosed in currently pending U.S. patent application Ser. No. 14/065,613 filed Oct. 29, 2013, to the inventor herein and wherein the content of this application is included herein in its entirety by reference. However, it is emphasized that other closure structures may be utilized and due to the preferred dimensional and structural characteristics of the introduction assembly 100 may also be introduced through the interior of the stabilizer catheter 122.

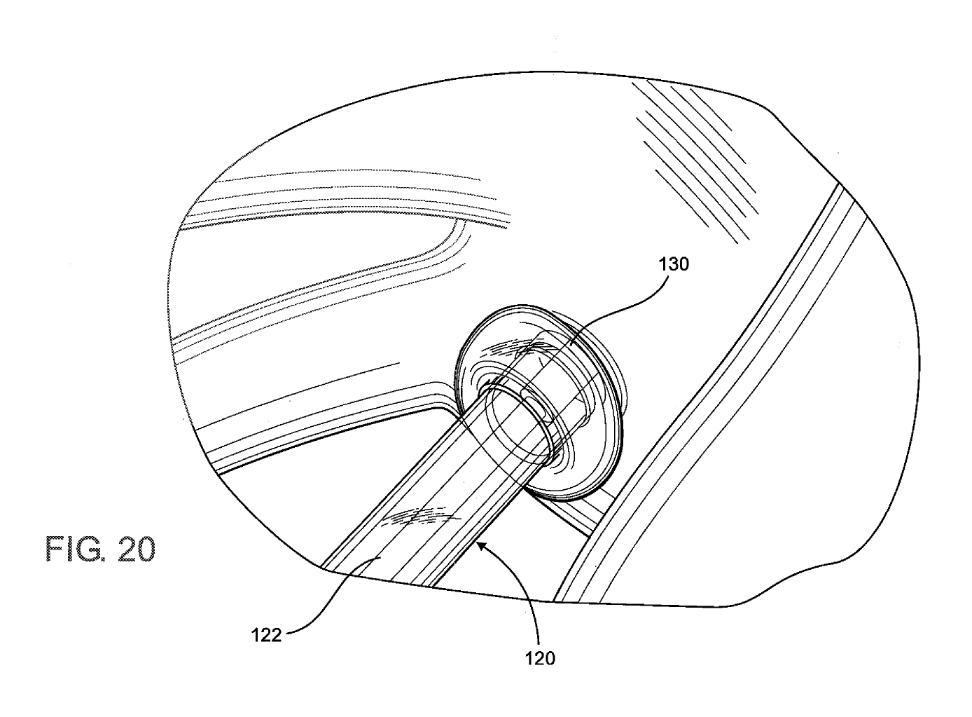

More specifically, the closure assembly 150 passes telescopically along the interior of the stabilizer catheter 122, through the entry site 111 and into the heart interior as represented in FIG. 19. Due to the inherent bias of each of the closure members 152 and 154, each will “automatically” expand into a sealing or closing orientation upon removal from the interior of the stabilizer catheter 122 and stabilizer structure 124 as also clearly represented in FIG. 19. Once the inner most closure member 152 is in the expanded, sealing orientation, the interior retaining member 128 of the retaining assembly 126 will be deflated to facilitate the removal of the stabilizer structure 124, as well as a positioning instrument 155, from the interior of the heart through the entry site 111. Concurrently the exterior retaining member 130 may be maintained in an inflated, retaining orientation as represented in FIG. 20.

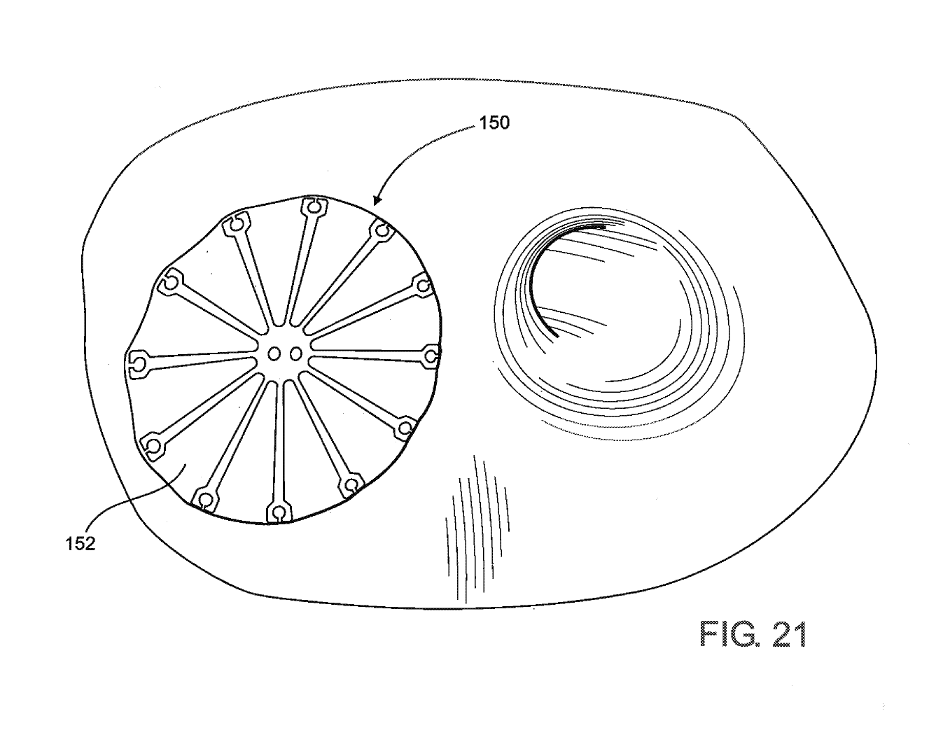

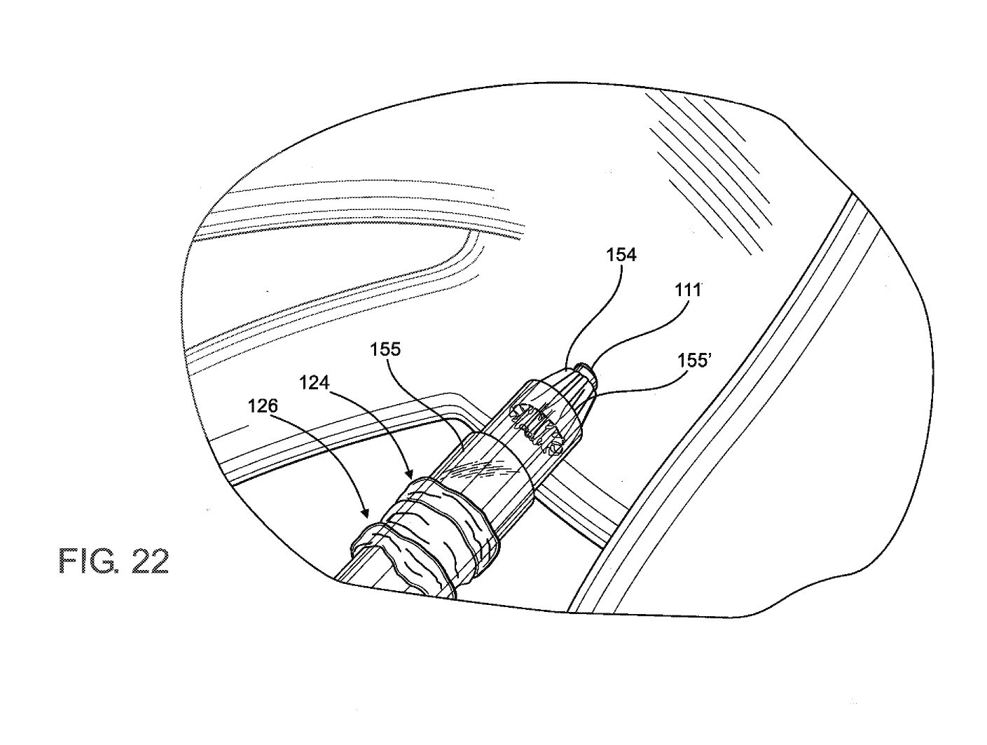

Upon removal of the stabilizer structure 124 from the entry site 111, the interior closure member 152 is brought into sealing or closing relation to the inner open end of the closure site 111 as represented in FIG. 21. Thereafter or substantially concurrently there with the positioning member 155 is withdrawn from the entry site 111, while the exterior closure member 154 remains on the exterior of the heart wall as represented in FIG. 22. Further withdrawal or removal of the stabilizer catheter 122, stabilizer structure 124 and positioning instrument 155 from the closure assembly 150 will result in the exterior closure member 154 passing out of the interior of the positioning member 155. This will result in the expansion of the exterior closure member 154 into a sealing, closing orientation with the exterior open end of the entry site 111, due to the inherent bias associated with its structure.

Accordingly, beneficial features of the closure assembly 150 and specifically including the first and second closing segments 152 and 154 include their formation from a material which has an at least minimal inherent bias. As such, both the first and second closing segments 152 and 154 may be at least initially disposed in a partially folded or otherwise collapsed orientation as they are disposed in and move along the interior of the positioning member 155. However, once passing out of the opening 155′ of the distal end of the positioning instrument 155 the “inherent bias” of the material of the first and second closing members 152 and 154 will facilitate their “automatic” expansion into the operative, sealing or closing orientation, as represented in FIGS. 19 and 21. Also of note is the possible forming of the first and second closing segments 82 and 84 from a material that may eventually dissolve on a timely basis by the exposure to ambient bodily fluids. The time in which the first and second closing segments 152 and 154 will be dissolved effectively coincides to the healing of the closed entry site 111.

Accordingly, the various above described embodiments of the introduction assembly and method of the present invention for the insertion of medical instruments through a thoracic passage into a targeted atrium or other portion of the heart are believed to overcome many of the disadvantages and complications associated with conventional or known related surgical procedures, as set forth above.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. An introduction assembly structured to facilitate insertion of instrumentation through the wall of the heart and into the interior thereof, said introduction assembly comprising:
an elongated sheath dimensioned for operative placement relative to the heart along a thoracic passage,
said sheath including open distal and proximal ends and a predetermined configuration along at least a portion of its length,
said predetermined configuration structured to facilitate orientation of said open distal end into said operative placement; said operative placement comprising a substantially flush, confronting relation of said open distal end to an exterior of the heart wall adjacent an entry site,
said sheath having a predetermined interior transverse dimensions sufficient to facilitate passage of the instrumentation there through into the heart, and
at least a portion of the length of said sheath formed of a rigid material, said rigid material having sufficient rigidity to facilitate manipulation of said sheath and disposition of said open distal end into said operative placement.

2. An introduction assembly as recited in claim 1 wherein said predetermined configuration comprises a curved configuration extending along a portion of the length of said sheath.

3. An introduction assembly as recited in claim 2 wherein said curved configuration is formed along a portion of the length of said sheath extending from said open distal end outwardly therefrom towards and in spaced relation to said open proximal end.

4. An introduction assembly as recited in claim 3 wherein said sheath is formed from a rigid material along at least a majority of its length.

5. An introduction assembly as recited in claim 3 wherein said sheath is formed of a rigid material along substantially its entire length.

6. An introduction assembly as recited in claim 1 wherein said sheath has a sufficient length for concurrent positioning of said open distal end in said operative placement and said open proximal end in an accessible location exteriorly of the thoracic cavity.

7. An introduction assembly as recited in claim 1 wherein said predetermined interior transverse dimension is greater than 7.3 mm.

8. An introduction assembly as recited in claim 7 wherein said predetermined interior transverse dimension of said sheath extends along the length of said sheath between said open proximal and distal ends.

9. An introduction assembly as recited in claim 1 further comprising a stabilizer assembly removably disposable within said sheath and including a stabilizer structure positionable through said open distal end into retaining relation to the entry site in the heart wall.

10. An introduction assembly as recited in claim 9 further comprising said stabilizer assembly including a central lumen having a predetermined transverse dimension sufficient to facilitate the passage there through of the instrumentation within the heart.

11. An introduction assembly as recited in 10 wherein said predetermined transverse dimension of said lumen is greater than 7.3 mm.

12. An introduction assembly as recited in claim 9 wherein said stabilizer structure extends through the entry site and comprises a retaining assembly disposed in a retaining orientation concurrently on an exterior and an interior of the heart wall.

13. An introduction assembly as recited in claim 12 wherein said retaining assembly comprises a central channel and two retaining members each disposed on different interior and exterior ends of said channel in said retaining orientation.

14. An introduction assembly as recited in claim 13 wherein each of said retaining members is inflatable into said retaining orientation; said retaining orientation comprising corresponding ones of said retaining members disposed in confronting relation with interior and exterior surfaces of the heart wall.

15. An introduction assembly as recited in claim 9 further comprising a dilator movably disposable within said stabilizer assembly and including a dilating tip dimensioned and configured to facilitate passage thereof into and expansion of the entry site.

16. An introduction assembly as recited in claim 15 wherein said dilating tip comprises a substantially pointed, partially conical configuration.

17. An introduction assembly as recited in claim 15 wherein said stabilizer structure is disposable relative to said dilator into said retaining relation with the entry site upon said expansion of the entry site.

18. An introduction assembly as recited in claim 15 further comprising a penetrating needle including a hollow interior, said needle movable through said sheath into penetrating relation with the heart wall at the entry site.

19. An introduction assembly as recited in claim 18 further comprising a guide wire dimensioned to pass through said hollow interior and into the heart interior concurrent to disposition of said penetrating needle through the entry site.

20. An introduction assembly as recited in claim 19 wherein said dilator is movably positioned on said guide wire into the heart through the entry site.

21. An introduction assembly as recited in claim 20 wherein said guide wire is removable from the within the heart through an interior of said dilator, concurrent to disposition of said dilator within the heart.

22. An introduction assembly as recited in claim 19 wherein said penetrating needle is movably disposed in telescoping relation on said guide wire and removal from the within the heart over said guide wire.

23. An introduction assembly as recited in claim 1 further comprising a camera mounted on said sheath and movable there with along the thoracic passage; said camera disposed of to define a field of vision of the entry site into the heart, when said open distal end is in said operative placement.

24. An introduction assembly as recited in claim 1 further comprising a buffer member connected to said sheath adjacent said open distal end, said buffer member disposed at and structured to restrict frictional confrontation of said open distal end with the exterior of the heart wall.

25. An introduction assembly structured to facilitate insertion of instrumentation into the heart through a wall thereof, said introduction assembly comprising:
an elongated sheath including an open distal end and a predetermined configuration extending along at least a portion of the length of said sheath,
said predetermined configuration disposed and structured to establish a substantially flush relation of said open distal end with an exterior of the heart wall, when said sheath is in said operative placement,
a stabilizer assembly movably disposable within said sheath and including a stabilizing structure positionable through said open distal end into a retaining orientation within an entry site in the heart wall,
said stabilizer assembly including a central lumen having a predetermined transverse dimension sufficient to facilitate the passage there through of instrumentation within the heart,
a dilator movably disposable within said stabilizer assembly and including a dilating tip dimensioned and configured to facilitate passage into and expansion of the entry site, and
said stabilizer structure disposable relative to said dilator into said retaining relation with the entry site upon the expansion of said entry site.

26. An introduction assembly as recited in claim 25 further comprising said stabilizer assembly and said stabilizer structure each including a central lumen having a predetermined transverse dimension sufficient to facilitate the passage there through of the instrumentation within the heart, said predetermined transverse dimension of said lumen is generally between 7.3 mm and 1.5 cm.

27. An introduction assembly as recited in claim 25 wherein said stabilizer structure extends through the entry site and comprises a retaining assembly and two retaining members, each of said two retaining members disposed on different interior and exterior ends of said stabilizing structure.

28. An introduction assembly as recited in claim 27 wherein each of said retaining members is independently inflatable into said retaining orientation; said retaining orientation comprising corresponding ones of said retaining members disposed in confronting relation with interior and exterior surfaces of the heart wall.

29. An introduction assembly as recited in claim 27 wherein said stabilizer structure is disposable relative to said dilator into said retaining orientation within the entry site upon said expansion of the entry site.
